(19) United States
(12) Patent Application Publication
(10) Pub. No.: US 2013/0045176 A1
(43) Pub. Date: Feb. 21, 2013

(54) STABLE PEPTIDE-PARTICLE ADDUCT COMPOSITIONS WITH IMPROVED SURFACE ADHESION

(71) Applicants: Xueping Jiang, Wilmington, DE (US); Yongqing Huang, Wilmington, DE (US)

(72) Inventors: Xueping Jiang, Wilmington, DE (US); Yongqing Huang, Wilmington, DE (US)

(73) Assignee: E. I. DU PONT DE NEMOURS AND COMPANY, Wilmington, DE (US)

(21) Appl. No.: 13/210,757

(22) Filed: Aug. 16, 2011

(51) Int. Cl. A61K 8/64; A61Q 5/06; C07K 14/00

(52) U.S. Cl. 424/70.6; 530/324

(57) ABSTRACT

Compositions and methods comprising the use of stabilized peptide-particulate benefit agent adducts are provided having a multi-block peptide component and a particulate benefit agent, where the multi-block peptide comprises the general structure A1-(S1)ₚ-(X1-Y)ₙ—(X2)ₘ-(S2)_{q}-A2 or A1-(S1)ₚ-(X1)ₘ-(Y—X2)ₚ-(S2)_{q}-A2; wherein, A1 and A2 are body surface-binding domains; S1 and S2 are optional peptide spacers; X1 and X2 are charged amino acid blocks; Y is a hydrophobic amino acid block comprising 3 to 10 contiguous hydrophobic amino acids; m is an integer ranging from 0 to 10; p and q are integers independently ranging from 0 to 3; and n is an integer ranging from 1 to 50. The stable adduct dispersion can be used to durably apply a particulate benefit agent to a body surface.

### FIELD OF THE INVENTION

The invention relates to the fields of personal care products, cosmetics and pharmaceuticals. More specifically, the invention relates to multi-domain or multi-block peptides and polypeptides that form complexes with particulate benefit agents, wherein the complexes are stably-dispersed and have improved surface adhesion to target materials.

### BACKGROUND OF THE INVENTION

Proteinaceous materials having strong affinity for a body surface have been used for targeted delivery of one or more personal care benefit agents. However, many of these materials used for targeted delivery are comprised or derived from immunoglobulins or immunoglobulin fragments (antibodies, antibody fragments, F_{ab}, single-chain variable fragments (scFv), and Camelidae V_{HH}) having affinity for the target surface. For example, Horikoshi et al. in JP 08104614 and Igarashi et al. in U.S. Pat. No. 5,597,386 describe hair coloring agents consisting of an anti-keratin antibody covalently attached to a dye or pigment. The antibody binds to the hair, thereby enhancing the binding of the hair coloring agent to the hair. Similarly, Kizawa et al. in JP 09003100 describe an antibody that recognizes the surface layer of hair and its use to treat hair. Terada et al. in JP 2002363026 describe the use of conjugates consisting of single-chain antibodies, preferably anti-keratin, coupled to dyes, ligands, and cosmetic agents for skin and hair care compositions. Although single-chain antibodies may be prepared using genetic engineering techniques, these molecules are expensive to prepare and may not be suitable for use in commercial personal care products due to their conserved structure (i.e. immunoglobulin folds) and large size.

Non-immunoglobulin derived scaffold proteins have also been developed for targeted delivery of benefit agents to a target surface, such as delivery of cosmetic agents to body surfaces (See Binz, H. et al. (2005) Nature Biotechnology 23, 1257-1268 for a review of various proteins used in scaffold-assisted binding). Findlay in WO 00/048558 describes the use of calycin-like scaffold proteins, such as β-lactoglobulin, which contain a binding domain for a cosmetic agent and another binding domain that binds to at least a part of the surface of a hair fiber or skin surface. Houtzager et al. in WO 03/050283 and US 2006-0140889 also describe affinity proteins having a defined core scaffold structure for controlled application of cosmetic substances. As with immunoglobulin-like proteins, these large scaffold proteins are somewhat limited by the requirement to maintain the underlying core structure for effective binding and are expensive to produce.

Short, single chain peptides (i.e., “target surface-binding peptides”) having affinity for a target surface can be identified and isolated from peptide libraries using any number of biopanning techniques well known to those skilled in the art including, but not limited to, bacterial display; yeast display, combinatorial solid phase peptide synthesis, phage display, ribosome display, and mRNA display technology (PROFUSION™; U.S. Pat. No. 6,258,558). The target surface-binding peptides identified using such techniques are typically no more than 60 amino acids in length and often have a binding affinity value (as reported as an MB₅₀ or K_{D} value) of 10⁻⁴ M or less for the surface of the target material. However, for some commercial applications the individual biopanned peptides may not provide the durability necessary to achieve the desired effect. The lack in durability may be especially evident when attempting to durably couple a particulate benefit agent to a target surface.

Single chain peptide-based reagents lacking a scaffold support or immunoglobulin fold have been developed that can be used to couple benefit agents to a target surface. Examples of target surfaces include, but not are limited to body surfaces such as hair, skin, nail, and teeth (U.S. Pat. Nos. 7,220,405; 7,309,482; and 7,285,264; and U.S. Patent Application Publication Nos. 2005-0226839; 2007-0196305; 2006-0199206; 2007-0065387; 2008-0107614; 2007-0110686; and 2006-0073111; and published PCT applications WO2008/054746; WO2004/048399; and WO2008/073368) as well as other surfaces such as pigments and miscellaneous print media (U.S. Patent Application Publication No. 2005-0054752), and various polymers such as polymethylmethacrylate (U.S. Pat. No. 7,858,581), polypropylene (U.S. Patent Application Publication No. 2007-0264720), nylon (U.S. Pat. No. 7,709,601 and U.S. Patent Application Publication No. 2003-0185870), polytetrafluoroethylene (U.S. Pat. No. 7,700,716), polyethylene (U.S. Patent Application Publication No. 2007-0141628), and polystyrene (U.S. Pat. No. 7,632,919). However, some single chain peptide-based reagents may lack the durability required for certain commercial applications, especially when coupling a particulate benefit agent to a body surface in a highly stringent matrix.

Many of the previously disclosed peptide-based reagents and pigments were applied sequentially to the body surface to achieve the desired effect (such as hair coloring). However, commercial cosmetic products comprising a two component system for sequential treatment may increase the time and cost of producing such products and is generally considered less attractive to the common consumer.

Several patent applications disclose the application of a peptide reagent and the benefit agent concomitantly to a body surface (U.S. Patent Application Publication Nos. 2007-0067924 and 2007-0065387; U.S. Pat. No. 7,285,264; and International Patent Application Publication NO. WO2008/054746). However, the described process often involves mixing the two individually packaged components shortly before or concomitantly with application to the body surface. This may require a personal care system having multiple chambers/bottles to keep the reagents separated until use. The use of a multi-chambered bottles or compartments is less attractive due to the packaging costs and may not be possible for certain personal care applications.

It is generally assumed that the use of a personal care product that does not require the mixing of multiple components by the consumer is more attractive product offering. As such, it is desirable to provide a personal care system wherein the particulate benefit agent and the peptide-based reagent are pre-formed into a stably dispersed peptide-particulate benefit agent adduct. The stably dispersed adduct can then be applied to the body surface to effective delivery the benefit agent to the body surface.

Therefore, a problem to be solved is to provide a stable dispersion of a peptide-particulate benefit agent adduct suitable for use in personal care compositions, as well as methods for preparing the same. In addition, the peptide-particulate benefit agent adducts should demonstrate a more durable binding to withstand common stresses associated with exposure to detergents and surfactants such as washing, shampooing, brushing one's teeth, laundering and the like.

### SUMMARY OF THE INVENTION

The problems described above have been addressed by preparation of a peptide-particulate benefit agent adduct demonstrating unexpectedly superior binding properties when applied to a body surface. For example, in one embodiment, the inventive peptide-particulate benefit agent adduct provides superior retention of a benefit agent on a body surface as determined by resistance to treatments with detergents and/or surfactants, e.g., shampoo treatment, washing, laundering, and the like.

In another embodiment, the peptide-particulate benefit agent adducts provide for more stable carrier systems (e.g., dispersions, emulsions, microemulsions, lotions, creams, gels, and the like) due to the peptide-particulate benefit agent adduct achieving a more beneficial zeta potential in aqueous media. Of particular relevance is that the peptide-particulate benefit agent adducts described herein comprise particulate benefit agents, which generally have a tendency to aggregate (e.g., coagulate or flocculate). Therefore, the peptide-particulate benefit agent adducts encompassed by this disclosure provide unexpectedly superior stability of compositions comprising particulate benefit agents.

In an additional embodiment, the peptide-particulate benefit agent adduct described herein comprises a multi-block peptide (MBP) associated with a particulate benefit agent wherein each block of the MBP contributes to the stability and functionality of the peptide-particulate benefit agent adduct.

In one embodiment, a peptide-particulate benefit agent adduct is provided comprising:

a) a particulate benefit agent; and

b) a peptide of having the general structure of

A1-(S1)ₚ-(X1-Y)ₚ-(X2)ₘ-(S2)_{q}-A2 or

A1-(S1)ₚ-(X1)ₘ-(Y—X2)ₚ-(S2)_{q}-A2
wherein,

A1 and A2 are binding domains having affinity to a body surface; wherein both A1 and A2 independently consist of 1 to 3 body surface-binding peptides (BSBP); each BSBP independently ranging from 7 to 60 amino acids in length and have affinity for the same body surface;

S1 and S2 are optional peptide spacers comprising 1 to 30 amino acids in length wherein the spacers contain less than 30 mol % charged amino acids

X1 and X2 are charged amino acid blocks; wherein X1 and X2 do not consist of net opposite charges; wherein X1 and X2 are independently 6 to 36 amino acids in length having 3 to 18 charged amino acids;

Y is a hydrophobic amino acid block comprising 3 to 10 contiguous hydrophobic amino acids;

m is an integer ranging from 0 to 10;

p and q are integers independently ranging from 0 to 3; and

n is an integer ranging from 1 to 50; and

wherein average particle size of the peptide-particulate benefit agent adduct is between 0.010 μm and 75 μm

In one aspect, the charged amino acid blocks X1 and X2 both have a net positive charge.

In another aspect, the charged amino acid blocks X1 and X2 both have a net negative charge.

A stable dispersion comprising the peptide-particulate benefit agent adduct is also provided. In one aspect, the stable dispersion is charged stabilized. In a preferred embodiment, the absolute value of the zeta potential of the stably dispersed peptide-particulate benefit agent adduct is at least 20 mV.

In another aspect, a method of forming a charge stabilized peptide-particulate benefit agent adduct is provided comprising,

a) providing
1) a particulate benefit agent having average particle size between 0.010 μm and 75 μm;
2) the peptide of as described above;

b) contacting the particulate benefit agent and the peptide in an aqueous medium under conditions suitable for forming a peptide-particulate benefit agent adduct; and

c) altering the pH of the aqueous medium until the absolute value of the zeta potential of the peptide-particulate benefit agent adduct is at least 20 mV.

In another embodiment, a method of applying a benefit agent to a body surface is provided comprising,

a) contacting a body surface with a composition comprising a population of the peptide-particulate benefit agent adduct as described above under conditions whereby a portion of the population of the peptide-particulate benefit agent adduct durably binds non-covalently to the body surface;

b) optionally, washing the body surface to remove non-durably bound peptide-particulate benefit agent adduct from the body surface; and

c) optionally repeating steps (a) and (b).

In a further aspect of the above method, a cationic polymer may also be applied once a desired amount of peptide-particulate benefit agent adducts are bound to the body surface.

In another embodiment, the method of applying a benefit agent to a body surface uses a mixture of peptide-particular benefit agents comprising different pigment or colorant particles may be used to achieve the desired coloration.

In another embodiment, the particulate benefit agent may comprise a pigment or other coloring agent that is desired for a particular body surface.

In yet another embodiment, the particulate benefit agent may comprise a fragrance or aromatic composition.

In yet another embodiment, the particulate benefit agent may comprise a ultraviolet radiation disperser, reflector, blocker or absorber.

In yet another embodiment, the particulate benefit agent may comprise a body surface conditioning composition. In such embodiments the particulate benefit agent may be a conditioner, moisturizer, emollient and the like, or a combination thereof.

In a further embodiment, the invention comprises an adduct composition and a method of using the adduct composition for applying a particulate benefit agent in a manner that resists removal from the body surface by washing, wetting, rinsing, conditioning, bathing, drying, exposure to sun, wind, rain and the like. Thus, the invention encompasses a method of enhancing the amount of benefit agent retained on a body surface.

The peptide-particulate benefit agent adduct can be formulated into various types of compositions, including but not limited to solutions, dispersions, lotions, creams, gels, emulsions, microemulsions, nanoemulsions, and the like.

It is further recognized that the peptide-particulate benefit agent adduct provides multiphasic compositions having improved stability for a more useful shelf life. It is therefore an additional embodiment of the invention to provide a composition comprising the peptide-particulate benefit agent adduct described herein, wherein the particles are stabilized in a liquid medium by adjusting the electrostatic properties of the composition. Thus, in one such embodiment, the zeta potential of the composition is adjusted to a value that is less than about −20 mV or more than about +20 mV (i.e., “±20 my”); more preferably ±30 mV and even more preferably ±40 mV.

### BRIEF DESCRIPTION OF THE BIOLOGICAL SEQUENCES

The following sequences conform with 37 C.F.R. 1.821 1.825 (“Requirements for Patent Applications Containing Nucleotide Sequences and/or Amino Acid Sequence Disclosures—the Sequence Rules”) and are consistent with World Intellectual Property Organization (WIPO) Standard ST.25 (1998) and the sequence listing requirements of the EPO and PCT (Rules 5.2 and 49.5(a bis), and Section 208 and Annex C of the Administrative Instructions). The symbols and format used for nucleotide and amino acid sequence data comply with the rules set forth in 37 C.F.R. §1.822.

SEQ ID NOs: 1, 3-59, 63, 65-95 are the amino acid sequence of a hair-binding peptides. SEQ ID NO: 63 is the amino acid sequence of a cysteine-attached hair-binding peptide. SEQ ID NOs: 65-67 are the amino acid sequence of shampoo-resistant hair-binding peptides. SEQ ID NOs: 68-71 are the amino acid sequences of biotinylated hair-binding and skin-binding peptides. SEQ ID NO: 72 is the amino acid sequence of a hair conditioner resistant hair-binding peptide.

SEQ ID NOs: 2, 61, 96-101 are the amino acid sequences of a skin-binding peptide.

SEQ ID NO: 53 is the amino acid sequence of a hair-binding and nail-binding peptide.

SEQ ID NO: 60 is the amino acid sequence of a nail-binding peptide.

SEQ ID NO: 62 is the oligonucleotide primer used to sequence phage DNA.

SEQ ID NO: 64 is the amino acid sequence of the Caspase-3 cleavage site.

SEQ ID NOs: 102-106 are the amino acid sequences of empirically generated hair and skin-binding peptides.

SEQ ID NOs: 107-131 are the amino acid sequences of pigment-binding peptides.

SEQ ID NOs: 132-134 are the amino acid sequences of peptide spacers.

SEQ ID NOs: 135-138 are the amino acid sequences of hair conditioner and shampoo resistant hair-binding peptides.

SEQ ID NOs: 139-176 are the amino acid sequences of various silica-binding peptides.

SEQ ID NOs: 177-197 are the amino acid sequences of multi-block particulate benefit agent binding peptides, especially directed to particulate pigments.

SEQ ID NOs: 198 and 199 are the amino acid sequences of rationally designed pigment-binding domains.

### DETAILED DESCRIPTION OF THE INVENTION

A multi-block peptide is provided for forming stable peptide-particulate benefit agent adducts. The term “peptide-particulate benefit agent adduct” refers to a complex formed by the non-covalent binding of a particulate benefit agent to a multi-block peptide. As used herein, the term “multi-block” refers to the modular structure of the peptide-component of the peptide-particulate benefit agent adducts. The entire multi-block peptide comprises the “peptide component” of a given peptide-particulate benefit agent adduct. The peptide component, associated with a particulate benefit agent comprises the peptide-particulate benefit agent adduct or simply the “adduct.” In one embodiment, a complete peptide-particulate benefit agent adduct is provided, that is, the complex formed between the peptide component and the particulate benefit agent.

A method of preparing a stabilized composition is also provided comprising the peptide-particulate benefit agent adduct is encompassed herein. In this regard, the term “stabilized” or “stable” refers to the fact that a dispersion of peptide-particulate benefit agent adducts displays minimal agglomeration or aggregation at the end of test period. This is assessed by determining the volume-based median particle diameter, i.e. D₅₀, at day 0 (the day of preparing the dispersion) and day 7, an arbitrary endpoint of the test period. In the present application, a stable dispersion is one wherein the D₅₀ does not increase by more than 50% by day 7.

A method of enhancing the retention of a benefit agent is further encompassed by the invention. The use of the peptide-particulate benefit agent adduct described herein provides a more rigorous association between a body surface and a particulate benefit agent. This enhancement in retention is made evident by comparing the amount of benefit agent retained after washing or shampooing the body surface which has been contacted with either the peptide-particulate benefit agent adduct or the particulate benefit agent alone, i.e., without the peptide component. The amount of benefit agent retained after washing or shampooing is characteristically greater than the amount retained when the particulate benefit agent is applied alone, i.e., without the peptide component.

The following additional definitions are used herein and should be referred to for interpretation of the claims and the specification.

As used herein, the articles “a”, “an”, and “the” preceding an element or component of the invention are intended to be nonrestrictive regarding the number of instances (i.e., occurrences) of the element or component. Therefore “a”, “an”, and “the” should be read to include one or at least one, and the singular word form of the element or component also includes the plural unless the number is obviously meant to be singular.

As used herein, the term “comprising” means the presence of the stated features, integers, steps, or components as referred to in the claims, but that it does not preclude the presence or addition of one or more other features, integers, steps, components or groups thereof. The term “comprising” is intended to include embodiments encompassed by the terms “consisting essentially of” and “consisting of”. Similarly, the term “consisting essentially of” is intended to include embodiments encompassed by the term “consisting of”.

As used herein, the term “about” modifying the quantity of an ingredient or reactant employed refers to variation in the numerical quantity that can occur, for example, through typical measuring and liquid handling procedures used for making concentrates or use solutions in the real world; through inadvertent error in these procedures; through differences in the manufacture, source, or purity of the ingredients employed to make the compositions or carry out the methods; and the like. The term “about” also encompasses amounts that differ due to different equilibrium conditions for a composition resulting from a particular initial mixture. Whether or not modified by the term “about”, the claims include equivalents to the quantities.

Where present, all ranges are inclusive and combinable. For example, when a range of “1 to 5” is recited, the recited range should be construed as including ranges “1 to 4”, “1 to 3”, “1-2”, “1-2 & 4-5”, “1-3 & 5”, and the like.

As used herein, “contacting” refers to placing a composition in contact with the target body surface for a period of time sufficient to achieve the desired result (e.g., target surface binding of the peptide-particulate benefit agent adduct). Contacting includes spraying, treating, immersing, flushing, pouring on or in, mixing, combining, painting, coating, applying, affixing to and otherwise communicating a composition with the body surface.

As used herein, “BSBP” means body surface-binding peptide. In one aspect, the body surface binding peptide is a peptide having strong affinity for a body surface, such as hair, skin, nail, teeth, and tissues of the oral cavity, such as gums.

As used herein, “HBP” means hair-binding peptide.

As used herein, “SBP” means skin-binding peptide.

As used herein, “NBP” means nail-binding peptide.

As used herein, “TBP” means tooth-binding peptide.

As used herein, “OBP” means oral cavity surface-binding peptide.

As used herein, “Spacer” means a peptide spacer.

As used herein, the term “present invention” or “invention” as used herein is meant to apply generally to all embodiments of the invention as recited in the claims as presented or as later amended and supplemented.

As used herein, the term “peptide” refers to two or more amino acids joined to each other by peptide bonds or modified peptide bonds.

As used herein, the term “body surface” refers to any surface of the mammalian body that may serve as a substrate for the binding of the peptide-particulate benefit agent adducts. In a preferred aspect, the body surface is a human body surface. In another aspect, the body surface is a body surface comprising keratin, such as hair, skin, and nail. Typical body surfaces may include, but are not limited to hair, skin, nails, teeth, and tissues of the oral cavity, such as gums.

As used herein, the term “hair” as used herein refers to any type of mammalian hair, including non-facial body hair, hair on the head, eyebrows, eyelashes, and other facial hair. In a preferred aspect, the term hair refers to human hair.

As used herein, the term “skin” as used herein refers to mammalian skin, human skin, or substitutes for mammalian skin, such as pig skin, VITRO-SKIN® and EPIDERM™. In a preferred aspect, the term “skin” refers to human skin. Skin as a body surface will generally comprise a layer of epithelial cells and may additionally comprise a layer of endothelial cells.

As used herein, the term “nails” as used herein refers to mammalian nail tissue. In a preferred aspect the term “nails” refers to human fingernails and toenails.

As used herein, the term “tooth surface” will refer to a surface comprised of tooth enamel (typically exposed after professional cleaning or polishing) or tooth pellicle (a surface comprising salivary glycoproteins). Hydroxyapatite may be coated with salivary glycoproteins to mimic a natural tooth pellicle surface and may also be used for the identification of tooth-binding peptides (tooth enamel is predominantly comprised of hydroxyapatite).

As used herein, the term “oral cavity surface-binding peptide” refers to a peptide that binds with strong affinity to surfaces such as teeth, gums, cheeks, tongue, or other surfaces in the oral cavity. In one embodiment, the oral cavity surface-binding peptide is a peptide that binds with strong affinity to a tooth surface.

As used herein, the term “tooth-binding peptide” (TBP) will refer to a peptide that binds with strong affinity to tooth enamel and/or tooth pellicle. Examples of biopanned tooth-binding peptides (“fingers”) have been disclosed in U.S. Patent Application Publication Nos. 2008-0280810; 2010-0247457; and 2010-0247589, and U.S. Pat. No. 7,807,141. The tooth-binding fingers may be linked together (through an optional spacer) to form tooth-binding domains (“hands”).

As used herein, the terms “pellicle” and “tooth pellicle” will refer to the thin film (typically about 20 nm to about 200 μm in thickness) derived from salivary glycoproteins which forms over the surface of the tooth crown. Daily tooth brushing tends to only remove a portion of the pellicle surface while abrasive tooth cleaning and/or polishing will typically exposure more of the tooth enamel surface.

As used herein, the terms “enamel” and “tooth enamel” will refer to the highly mineralized tissue which forms the outer layer of the tooth. The enamel layer is composed primarily of crystalline calcium phosphate (i.e., hydroxyapatite) along with water and some organic material.

As used herein, the term “particulate benefit agent’ is a general term, relating to a particulate substance, which when applied to a body surface provides a beneficial effect. In one embodiment, the beneficial effect is a cosmetic or prophylactic effect. Particulate benefit agents may include sunscreen agents, conditioning agents, encapsulated fragrances, antimicrobial agents, antidandruff agents, antifungal agents, odor control agents, encapsulated bioactive agents, hair removal agents, anti-acne agents, and coloring agents, such as a pigment, a lake, a colored particle or a combination thereof. Active agents (bioactive agents) may be encapsulated or incorporated into particles for delivery.

As used herein, the terms “coupling” and “coupled” as used herein, refer to any chemical association and includes both covalent and non-covalent interactions.

As used herein, the term “stringency” as it is applied to the selection of the body-surface-binding peptides, refers to the concentration of the eluting agent (usually detergent) used to elute peptides from the body surface. Higher concentrations of the eluting agent provide more stringent conditions.

As used herein, the term “peptide-body surface complex” means structure comprising a peptide bound to a sample of a body surface via a binding site on the peptide.

As used herein, the term “MB₅₀” refers to the concentration of the binding peptide that gives a signal that is 50% of the maximum signal obtained in an ELISA-based binding assay (see, e.g., Example 3 of U.S. Patent Application Publication 2005-0022683; hereby incorporated by reference). The MB₅₀ provides an indication of the strength of the binding interaction or affinity of the components of the complex. The lower the value of MB₅₀, the stronger the interaction of the peptide has with its corresponding substrate.

As used herein, the term “binding affinity” refers to the strength of the interaction of a binding peptide with its respective substrate. The binding affinity is defined herein in terms of the MB₅₀ value, determined in an ELISA-based binding assay.

As used herein, the term “nanoparticles” are herein defined as particles with an average particle diameter of between 1 and 200 nm. Preferably, the average particle diameter of the particles is between about 1 and 40 nm. As used herein, “particle size” and “particle diameter” have the same meaning. Nanoparticles include, but are not limited to, metallic, semiconductor, polymer, or silica particles.

The term “amino acid” refers to the basic chemical structural unit of a protein or polypeptide. The following abbreviations are used herein to identify specific amino acids:

| | Three-Letter | One-Letter |
|---|---|---|
| Amino Acid | Abbreviation | Abbreviation |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |
| Any naturally-occurring amino acid | Xaa | X |
| (or as defined by the formulas | | |
| described herein) | | |

“Gene” refers to a nucleic acid fragment that expresses a specific protein, including regulatory sequences preceding (5′ non-coding sequences) and following (3′ non-coding sequences) the coding sequence. “Native gene” refers to a gene as found in nature with its own regulatory sequences “Chimeric gene” refers to any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. A “foreign” gene refers to a gene not normally found in the host organism, but that is introduced into the host organism by gene transfer. Foreign genes can comprise native genes inserted into a non-native organism, or chimeric genes. “Synthetic genes” can be assembled from oligonucleotide building blocks that are chemically synthesized using procedures known to those skilled in the art. These building blocks are ligated and annealed to form gene segments which are then enzymatically assembled to construct the entire gene. “Chemically synthesized”, as related to a sequence of DNA, means that the component nucleotides were assembled in vitro. Manual chemical synthesis of DNA may be accomplished using well-established procedures, or automated chemical synthesis can be performed using one of a number of commercially available machines. Accordingly, the genes can be tailored for optimal gene expression based on optimization of nucleotide sequence to reflect the codon bias of the host cell. The skilled artisan appreciates the likelihood of successful gene expression if codon usage is biased towards those codons favored by the host. Determination of preferred codons can be based on a survey of genes derived from the host cell where sequence information is available.

“Coding sequence” refers to a DNA sequence that codes for a specific amino acid sequence. “Suitable regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, introns, polyadenylation recognition sequences, RNA processing sites, effector binding sites and stem-loop structures.

“Promoter” refers to a DNA sequence capable of controlling the expression of a coding sequence or functional RNA. In general, a coding sequence is located 3′ to a promoter sequence. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental or physiological conditions. Promoters which cause a gene to be expressed in most cell types at most times are commonly referred to as “constitutive promoters”. It is further recognized that since in most cases the exact boundaries of regulatory sequences have not been completely defined, DNA fragments of different lengths may have identical promoter activity.

The term “expression”, as used herein, refers to the transcription and stable accumulation of sense (mRNA) or antisense RNA derived from the nucleic acid fragment of the invention. Expression may also refer to translation of mRNA into a polypeptide.

As used herein, the term “transformation” refers to the transfer of a nucleic acid fragment into the genome of a host organism, resulting in genetically stable inheritance. Host organisms containing the transformed nucleic acid fragments are referred to as “transgenic” or “recombinant” or “transformed” organisms.

As used herein, the term “host cell” refers to cell which has been transformed or transfected, or is capable of transformation or transfection by an exogenous polynucleotide sequence.

As used herein, the terms “plasmid”, “vector” and “cassette” refer to an extra chromosomal element often carrying genes which are not part of the central metabolism of the cell, and usually in the form of circular double-stranded DNA molecules. Such elements may be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′untranslated sequence into a cell. “Transformation cassette” refers to a specific vector containing a foreign gene and having elements in addition to the foreign gene that facilitate transformation of a particular host cell. “Expression cassette” refers to a specific vector containing a foreign gene and having elements in addition to the foreign gene that allow for enhanced expression of that gene in a foreign host.

As used herein, the term “phage” or “bacteriophage” refers to a virus that infects bacteria. Altered forms may be used for the purpose of the present invention. The preferred bacteriophage is derived from the “wild” phage, called M13. The M13 system can grow inside a bacterium, so that it does not destroy the cell it infects but causes it to make new phages continuously. It is a single-stranded DNA phage.

The term “phage display” refers to the display of functional foreign peptides or small proteins on the surface of bacteriophage or phagemid particles. Genetically engineered phage may be used to present peptides as segments of their native surface proteins. Peptide libraries may be produced by populations of phage with different gene sequences.

“PCR” or “polymerase chain reaction” is a technique used for the amplification of specific DNA segments (U.S. Pat. Nos. 4,683,195 and 4,800,159).

### Particulate Benefit Agents

The peptide-particulate benefit agent adduct of the invention may be formed in conjunction with a wide variety of particulate benefit agents known in the art of personal care. Examples of particulate benefit agents may include, but are not limited to, pigments, particulate conditioning agents, and inorganic sunscreens. In one embodiment, the particulate benefit agent is a sunscreen agent, conditioning agent, an encapsulated fragrance, an antimicrobial agent, an antidandruff agent, an antifungal, an odor control agent, an encapsulated bioactive agent, a hair removal agent, an anti-acne agent, or a coloring agent. In a further aspect, the coloring agent is a pigment, a colored particle or a combination thereof.

Particulate benefit agent and/or the peptide-particulate benefit agent adduct may range in size. In one embodiment, the average particle size of particulate benefit agent or the peptide-particulate benefit agent adduct ranges from 10 nm to 75 μm, preferably 10 nm to 10 μm, more preferably 100 nm to 5 μm, and most preferably 100 nm to 1 μm,

Non-particulate benefit agents (e.g., fragrances, antifungal agents, bioactive agents, and the like) may be incorporated (encapsulated, coated, absorbed, etc.) on or in a particulate carrier. In one embodiment, the particulate carrier is a mesoporous particle, such as mesoporous silica particles. Hollow porous silica particles suitable for delivery of an encapsulated or absorbed benefit agent may be prepared by using any number of well known methods (see U.S. Pat. No. 5,024,826 to Linton, H.; and U.S. Pat. No. 6,221,326 to Amiche, F., each herein incorporated by reference in its entirety). The porous silica shells typically have an average particle size ranging from 20 nm to 15 μm, a pore size ranging from 3 nm to 10 nm, a shell thickness ranging from 2 nm to 50 nm, and a specific surface of 25-400 m²/g. As such, many different materials may be incorporated into the particulate benefit agents subsequently used in the preparation of the present peptide-particulate benefit agent adducts.

As used herein, the term “pigment” means an insoluble or particulate colorant. It is a material that changes the color of light it reflects as the result of selective color absorption. A wide variety of organic and inorganic pigments alone or in combination may be used in the present invention. For example, distinct pigment particles may be prepared by combining within the same particle. Alternatively, distinct pigment particles may be combined as a mixture (e.g., dispersion), wherein the ratios of the distinct particles may be conveniently varied so as to provide varying shades of a particular color. In a further embodiment, a mixtures of peptide-particulate benefit agent adducts comprising 2 or more pigments or colored particles are used to achieve the desired coloration.

Pigments for coloring body surfaces are well known in the art (see for example Green et al. (WO 0107009), CFTA International Color Handbook, 2nd ed., Micelle Press, England (1992) and Cosmetic Handbook, US Food and Drug Administration, FDA/IAS Booklet (1992)), and are available commercially from various sources (for example Bayer, Pittsburgh, Pa.; Ciba-Geigy, Tarrytown, N.Y.; ICI, Bridgewater, N.J.; Sandoz, Vienna, Austria; BASF, Mount Olive, N.J.; and Hoechst, Frankfurt, Germany). Exemplary pigments include, but are not limited to, D&C Red No. 36, D&C Red No. 30, D&C Orange No. 17, Green 3 Lake, Ext. Yellow 7 Lake, Orange 4 Lake, and Red 28 Lake; the calcium lakes of D&C Red Nos. 7, 11, 31 and 34, the barium lake of D&C Red No. 12, the strontium lake D&C Red No. 13, the aluminum lakes of FD&C Yellow No. 5, of FD&C Yellow No. 6, of FD&C No. 40, of D&C Red Nos. 21, 22, 27, and 28, of FD&C Blue No. 1, of D&C Orange No. 5, of D&C Yellow No. 10, the zirconium lake of D&C Red No. 33; CROMOPHTHAL® Yellow 131AK (Ciba Specialty Chemicals), SUNFAST® Magenta 122 (Sun Chemical) and SUNFAST® Blue 15:3 (Sun Chemical), iron oxides, calcium carbonate, aluminum hydroxide, calcium sulfate, kaolin, ferric ammonium ferrocyanide, magnesium carbonate, carmine, barium sulfate, mica, bismuth oxychloride, zinc stearate, manganese violet, chromium oxide, titanium dioxide, black titanium dioxide, titanium dioxide nanoparticles, zinc oxide, barium oxide, ultramarine blue, bismuth citrate, and white minerals such as hydroxyapatite, and Zircon (zirconium silicate), and carbon black particles.

Pigments are substantially insoluble and therefore, are used in dispersed form. The pigment may be dispersed using a dispersant or a self-dispersing pigment may be used. When a dispersant is used to disperse the pigment, the dispersant may be any suitable dispersant known in the art, including, but not limited to, random or structured organic polymeric dispersants, as described below; protein dispersants, such as those described by Brueckmann et al. (U.S. Pat. No. 5,124,438); and peptide-based dispersants, such as those described by O'Brien et al. (U.S. Patent Application Publication No. 2005-0054752). Preferred random organic polymeric dispersants include acrylic polymer and styrene-acrylic polymers. Most preferred are structured dispersants, which include AB, BAB and ABC block copolymers, branched polymers and graft polymers. Preferably the organic polymers comprise monomer units selected from the group consisting of acrylate, methacrylate, butyl methacrylate, 2-ethylhexyl methacrylate, benzyl methacrylate, phenoxyethyl acrylate, ethoxytriethyleneglycol methacrylate, polyethylene glycol methacrylate, polyethylene glycol acrylate, acrylic acid, methacrylic acid, methacrylamide, acrylamide, dimethylaminoethyl methacrylate, hydroxyethyl acrylate, and hydroxyethyl methacrylate, such as those described by Nigan (U.S. Patent Application Publication No. 2004-0232377). Some useful structured polymer dispersants are disclosed in U.S. Pat. Nos. 5,085,698 and 5,231,131 (the disclosures of which are incorporated herein by reference) and EP-A-0556649. Additionally, pigments may be dispersed using a surface active agent comprising lignin sulfonic acids and a polypeptide, as described by Cioca et al. in U.S. Pat. No. 4,494,994, which is incorporated herein by reference.

The pigment may optionally be surface-treated prior to coating with organic polymer. Common surface treatments include, but are not limited to, alkyl silane, siloxane, methicone, and dimethicone. Surface treatment increases the range of polymers that have an affinity for the pigment surface.

Metallic and semiconductor nanoparticles may also be used as hair coloring agents due to their strong emission of light (Vic et al., U.S. Patent Application Publication No. 2004-0010864). The metallic nanoparticles include, but are not limited to, particles of gold, silver, platinum, palladium, iridium, rhodium, osmium, iron, copper, cobalt, and alloys composed of these metals. An “alloy” is herein defined as a homogeneous mixture of two or more metals. The “semiconductor nanoparticles” include, but are not limited to, particles of cadmium selenide, cadmium sulfide, silver sulfide, cadmium sulfide, zinc oxide, zinc sulfide, zinc selenide, lead sulfide, gallium arsenide, silicon, tin oxide, iron oxide, and indium phosphide. Methods for the preparation of stabilized, water-soluble metal and semiconductor nanoparticles are known in the art, and suitable examples are described by Huang et al. in U.S. Patent Application Publication No. 2004-0115345, which is incorporated herein by reference. The color of the nanoparticles depends on the size of the particles. Therefore, by controlling the size of the nanoparticles, different colors may be obtained.

The particulate benefit agent may also be an inorganic UV sunscreen, which absorbs, reflects, or scatters ultraviolet light at wavelengths from 290 to 400 nanometers. Inorganic UV sunscreen materials are typically inorganic pigments and metal oxides including, but not limited to, titanium dioxide (such as SunSmart available from BASF Corp., Berlin, Germany), zinc oxide, and iron oxide. A preferred sunscreen is titanium dioxide nanoparticles. Suitable titanium dioxide nanoparticles are described in U.S. Pat. Nos. 5,451,390; 5,672,330; and 5,762,914. Titanium dioxide P25 is an example of a suitable commercial product available from Degussa (Parsippany, N.J.). Other commercial suppliers of titanium dioxide nanoparticles include Kemira (Helsinki, Finland), Sachtleben (Duisburg, Germany) and Tayca (Osaka, Japan).

The titanium dioxide nanoparticles typically have an average particle size diameter of less than 100 nanometers (nm) as determined by dynamic light scattering which measures the particle size distribution of particles in liquid suspension. A process to prepare titanium dioxide nanoparticles is by injecting oxygen and titanium halide, preferably titanium tetrachloride, into a high-temperature reaction zone, typically ranging from 400 to 2000° C. Under the high temperature conditions present in the reaction zone, nanoparticles of titanium dioxide are formed having high surface area and a narrow size distribution. The energy source in the reactor may be any heating source such as a plasma torch.

### Body Surfaces

Body surfaces of the invention are any surface on the mammalian body that will serve as a substrate for a binding peptide. In a preferred aspect the body surfaces are any surface on a human body that will surface as a substrate for a binding peptide. Typical body surfaces include, but are not limited to hair, skin, nails, teeth, and the tissues of the oral cavity, such as gums, cheeks, and the like. In many cases the body surfaces of the invention will be exposed to air, however in some instances, the oral cavity for example, the surfaces will be internal. Accordingly body surfaces may include layers of both epithelial and well as endothelial cells.

Samples of body surfaces are available from a variety of sources. For example, human hair samples are available commercially, for example from International Hair Importers and Products (Bellerose, N.Y.), in different colors, such as brown, black, red, and blond, and in various types, such as African-American, Caucasian, and Asian. Additionally, the hair samples may be treated for example using hydrogen peroxide to obtain bleached hair. Human skin samples may be obtained from cadavers or in vitro human skin cultures. Additionally, pig skin, available from butcher shops and supermarkets, VITRO-SKIN®, available from IMS Inc. (Milford, Conn.), and EPIDERM™, available from MatTek Corp. (Ashland, Mass.), are good substitutes for human skin. Human fingernails and toenails may be obtained from volunteers. Extracted human teeth and false teeth may be obtained from dental offices. Additionally, hydroxyapatite, available in many forms for example from Berkeley Advanced Biomaterials, Inc. (San Leandro, Calif.), may be used as a model for human teeth.

### Body Surface-Binding Peptides

Body surface-binding peptides form the binding domain moieties A1 and A2. These binding peptides may be identical or different and are defined herein as peptide sequences that specifically bind with high affinity to specific body surfaces, including, but not limited to hair, nails, teeth, gums, skin, and the tissues of the oral cavity. Suitable body surface-binding peptide sequences may be selected using combinatorial methods that are well known in the art or may be empirically generated. The body surface binding peptides that comprise the A1 and A2 domains of the peptide component of the invention have a binding affinity for their respective substrate, as measured by MB₅₀ values, of less than or equal to about 10⁻²M, preferably less than or equal to about 10⁻³ M, more preferably less than or equal to about 10⁻⁴M, even more preferably less than or equal to about 10⁻⁶ M, even further preferably less than or equal to about 10⁻⁶M, and most preferably less than or equal to about 10⁻⁷ to about 10⁻⁸ M.

Combinatorially generated body surface-binding peptides of the present invention are from about 7 amino acids to about 60 amino acids, more preferably, from about 7 amino acids to about 25 amino acids in length. The body surface-binding peptides of the present invention may be generated randomly and then selected against a specific body surface, for example, hair, skin, nail, or tooth sample, based upon their binding affinity for the surface of interest. The generation of random libraries of peptides is well known and may be accomplished by a variety of techniques including, bacterial display (Kemp, D. J.; Proc. Natl. Acad. Sci. USA 78(7):4520-4524 (1981), and Helfman et al., Proc. Natl. Acad. Sci. USA 80(1):31-35, (1983)), yeast display (Chien et al., Proc Natl Acad Sci USA 88(21):9578-82 (1991)), combinatorial solid phase peptide synthesis (U.S. Pat. Nos. 5,449,754; 5,480,971; 5,585,275 and 5,639,603), and phage display technology (U.S. Pat. Nos. 5,223,409; 5,403,484; 5,571,698; and 5,837,500). Techniques to generate such biological peptide libraries are described in Dani, M., J. of Receptor & Signal Transduction Res., 21(4):447-468 (2001). Additionally, phage display libraries are available commercially from companies such as New England BioLabs (Beverly, Mass.).

A preferred method to randomly generate peptides is by phage display. Phage display is an in vitro selection technique in which a peptide or protein is genetically fused to a coat protein of a bacteriophage, resulting in display of fused peptide on the exterior of the phage virion, while the DNA encoding the fusion resides within the virion. This physical linkage between the displayed peptide and the DNA encoding it allows screening of vast numbers of variants of peptides, each linked to a corresponding DNA sequence, by a simple in vitro selection procedure called “biopanning”. In its simplest form, biopanning is carried out by incubating the pool of phage-displayed variants with a target of interest that has been immobilized on a plate or bead, washing away unbound phage, and eluting specifically bound phage by disrupting the binding interactions between the phage and the target. The eluted phage is then amplified in vivo and the process is repeated, resulting in a stepwise enrichment of the phage pool in favor of the tightest binding sequences. After 3 or more rounds of selection/amplification, individual clones are characterized by DNA sequencing.

More specifically, after a suitable library of peptides has been generated or purchased, the library is then contacted with an appropriate amount of the test substrate, specifically a body surface sample. The library of peptides is dissolved in a suitable solution for contacting the sample. The body surface sample may be suspended in the solution or may be immobilized on a plate or bead. A preferred solution is a buffered aqueous saline solution containing a surfactant. A suitable solution is Tris-buffered saline (TBS) with 0.5% TWEEN® 20. The solution may additionally be agitated by any means in order to increase the mass transfer rate of the peptides to body surface sample, thereby shortening the time required to attain maximum binding.

Upon contact, a number of the randomly generated peptides will bind to the body surface sample to form a peptide-body-surface complex, for example a peptide-hair, peptide-skin, peptide-nail, or peptide-tooth complex. Unbound peptide may be removed by washing. After all unbound material is removed, peptides having varying degrees of binding affinities for the test surface may be fractionated by selected washings in buffers having varying stringencies. Increasing the stringency of the buffer used increases the required strength of the bond between the peptide and body surface in the peptide-body surface complex.

A number of substances may be used to vary the stringency of the buffer solution in peptide selection including, but not limited to, acidic pH (1.5-3.0); basic pH (10-12.5); high salt concentrations such as MgCl₂ (3-5 M) and LiCl (5-10 M); water; ethylene glycol (25-50%); dioxane (5-20%); thiocyanate (1-5 M); guanidine (2-5 M); urea (2-8 M); and various concentrations of different surfactants such as SDS (sodium dodecyl sulfate), DOC (sodium deoxycholate), Nonidet P-40, Triton X-100, TWEEN® 20, wherein TWEEN® 20 is preferred. These substances may be prepared in buffer solutions including, but not limited to, Tris-HCl, Tris-buffered saline, Tris-borate, Tris-acetic acid, triethylamine, phosphate buffer, and glycine-HCl, wherein Tris-buffered saline solution is preferred. It will be appreciated that peptides having increasing binding affinities for body surface substrates may be eluted by repeating the selection process using buffers with increasing stringencies.

The eluted peptides can be identified and sequenced by any means known in the art. Thus, the following method for generating the body surface-binding peptides, for example, hair-binding peptides, skin-binding peptides, nail-binding peptides, or tooth-binding peptides, may be used. A library of combinatorially generated phage-peptides is contacted with the body surface of interest, to form phage peptide-body surface complexes. The phage-peptide-body-surface complex is separated from uncomplexed peptides and unbound substrate, and the bound phage-peptides from the phage-peptide-body surface complexes are eluted from the complex, preferably by acid treatment. Then, the eluted phage-peptides are identified and sequenced. To identify peptide sequences that bind to one substrate but not to another, for example peptides that bind to hair, but not to skin or peptides that bind to skin, but not to hair, a subtractive panning step is added. Specifically, the library of combinatorially generated phage-peptides is first contacted with the non-target to remove phage-peptides that bind to it. Then, the non-binding phage-peptides are contacted with the desired substrate and the above process is followed. Alternatively, the library of combinatorially generated phage-peptides may be contacted with the non-target and the desired substrate simultaneously. Then, the phage-peptide-body surface complexes are separated from the phage-peptide-non-target complexes and the method described above is followed for the desired phage-peptide-body surface complexes.

In one embodiment, a modified phage display screening method for isolating peptides with a higher affinity for body surfaces is used. In the modified method, the phage-peptide-body surface complexes are formed as described above. Then, these complexes are treated with an elution buffer. Any of the elution buffers described above may be used. Preferably, the elution buffer is an acidic solution. Then, the remaining, elution-resistant phage-peptide-body surface complexes are used to directly infect a bacterial host cell, such as E. coli ER2738. The infected host cells are grown in an appropriate growth medium, such as LB (Luria-Bertani) medium, and this culture is spread onto agar, containing a suitable growth medium, such as LB medium with IPTG (isopropyl β-D-thiogalactopyranoside) and S-GAL™. After growth, the plaques are picked for DNA isolation and are sequenced to identify the peptide sequences with a high binding affinity for the body surface of interest.

PCR may be used to identify the elution-resistant phage-peptides from the modified phage display screening method, described above, by directly carrying out PCR on the phage-peptide-body surface complexes using the appropriate primers, as described by Janssen et al. in U.S. Patent Application Publication No. 2003-0152976, which is incorporated herein by reference.

Hair-binding, skin-binding, and nail-binding peptides have been identified using the above methods, as described by Huang et al. in U.S. Patent Application Publication No. 2005-0050656, and U.S. Patent Application Publication No. 2005-0226839, both of which are incorporated herein by reference. Examples of hair-binding peptides are provided as SEQ ID NOs: 1, 3-59, 63, 65-95, 102-106, and 135-138. Examples of skin-binding peptides are provided as SEQ ID NOs: 2, 61, and 96-106. Examples of nail-binding peptides are provided as SEQ ID NOs: 53 and 60.

Alternatively, hair and skin-binding peptide sequences may be generated empirically by designing peptides that comprise positively charged amino acids, which can bind to hair and skin via electrostatic interaction, as described by Rothe et al. (WO 2004/000257). The empirically generated hair and skin-binding peptides have between about 7 amino acids to about 60 amino acids, preferably from about 7 to about 25 amino acids, and comprise at least about 40 mole % positively charged amino acids, such as lysine, arginine, and histidine. Peptide sequences containing tripeptide motifs such as HRK, RHK, HKR, RKH, KRH, KHR, HKX, KRX, RKX, HRX, KHX and RHX are most preferred where X can be any natural amino acid but is most preferably selected from neutral side chain amino acids such as glycine, alanine, proline, leucine, isoleucine, valine and phenylalanine. In addition, it should be understood that the peptide sequences must meet other functional requirements in the end use including solubility, viscosity and compatibility with other components in a formulated product and will therefore vary according to the needs of the application. In some cases the peptide may contain up to 60 mole % of amino acids not comprising histidine, lysine or arginine. Examples of empirically generated hair-binding and skin peptides may include, but are not limited to, SEQ ID NOs: 102-106.

### Multi-Block Peptides

Multi-block peptides are provided having the structure provided by the general structure:

A1-(S1)ₚ-(X1-Y)ₚ-(X2)ₘ-(S2)_{q}-A2 or

A1-(S1)ₚ-(X1)ₘ-(Y—X2)ₚ-(S2)_{q}-A2

wherein,

A1 and A2 are binding domains having affinity to a body surface; wherein both A1 and A2 independently consist of 1 to 3 body surface-binding peptides (BSBP); each BSBP independently ranging from 7 to 60 amino acids in length and have affinity for the same body surface;

S1 and S2 are optional peptide spacers comprising 1 to 30 amino acids in length wherein the spacers contain less than 30 mol % charged amino acids

X1 and X2 are charged amino acid blocks; wherein X1 and X2 do not consist of net opposite charges; wherein X1 and X2 are independently 6 to 36 amino acids in length having 3 to 18 charged amino acids;

Y is a hydrophobic amino acid block comprising 3 to 10 contiguous hydrophobic amino acids;

m is an integer ranging from 0 to 10;

p and q are integers independently ranging from 0 to 3; and

n is an integer ranging from 1 to 50.

The general structures provided above illustrate the arrangement of amino acid blocks, i.e., domains or modules, of the multi-block peptide component capable of forming a stable peptide-particulate benefit agent adduct. Each terminus of the peptide component comprises a target surface binding domain; wherein each binding domain (referred to as blocks/domains “A1” and “A2”) independently comprises 1 to 3 body surface-binding peptides (BSBPs); each body surface-binding peptides independently ranging form 7 to 60 amino acids in length and have affinity for the same type of body surface (hair, skin, nail, teeth, tissues of the oral cavity, and the like). In one embodiment, domains A1 and A2 bind to the respective body surface with an affinity greater than a control peptide domain (i.e., a domain that does not possess body-surface binding properties; or a domain or peptide that is not specifically generated or isolated based upon the peptide's demonstrable body-surface binding properties).

The A1 and A2 are directly or indirectly attached to the charged blocks of the particulate benefit agent binding block, X1 and X2, respectively, when m is 1 to 10. In some embodiments, either X1 or X2 may be absent (i.e. m=0) whereby the hydrophobic amino acid block Y is attached directly to an optional spacer or to the respective binding domain A1 or A2. In a preferred embodiment, m is equal to 1. X1 and X2 may have a net positive or net negative charge as along as both are positive or negative within the same peptide, assuming both X1 and X2 are present. In a preferred embodiment, both are positively charged. In one aspect, X1 and X2 will be have a net charge that is opposite that of the target particulate benefit agent. Direct attachment indicates that one or both of the optional spacer blocks shown in the above general structures (blocks “S1” and “S2”) are not present in the multi-block peptide component. In such cases each body surface-binding domain (A1 and A2) is directly bonded to the charged blocks, X1 and X2, of the multi-block peptide component.

The body surface-binding domains A1 and A2 can each be attached to its respective charged block (preferably positively charged block when using a particle having a negative surface charge) by an intervening spacer S1 and S2, respectively. In some embodiments, the spacer is a peptide block encoded by a recombinant heterologous DNA. One spacer peptide, S1, extends in frame from the carboxy terminus of “A1” to the amino terminus of charged amino acid block X1. A second spacer peptide, S2, extends from the carboxy terminus of a second charged block, X2, to the amino terminus of “A2” (spacer between A2 and the immediately upstream second charged block).

The charged blocks X1 and X2 can be distinct or identical so long as they do not have net opposite charges (i.e., one has a net positive charge while the other has a net negative charge). When X1 or X2 are repeated in the above general structures the exact composition of each repeating occurrence may vary in length and composition so long as the relative charge between the repeating units blocks remains similar (i.e., all occurrences have a net positive or net negative charge). Positioned in between X1 and X2 is a hydrophobic block Y, that comprises a stretch of 3 to 10 contiguous/continuous hydrophobic amino acids. The preferred hydrophobic amino acids are those having a hydrophobicity parameter of 0.8 or greater (see Table 2). In a preferred embodiment, X1, Y and X2 together comprise the particulate agent binding block (X1-Y)ₙ—(X2)ₘ or (X1)ₘ-(Y—X2)ₙ where m=1 to 10 and n=1 to 50. In a preferred aspect, m=1 and n=1 to 50, preferably 1 to 25, more preferably 1 to 10, and most preferably 1 to 3. In one embodiment, X1 or X2 may not be present (i.e., m=0) such that the particulate agent binding block may comprise of one or more of blocks of (X1-Y)ₙ or (Y—X2)ₙ, where n=1 to 50.

### The Particulate Benefit Agent-Binding Domain

X1, Y and X2 together comprise the particulate agent binding block (X1-Y)ₙ—(X2)ₘ or (X1)ₘ-(Y—X2)ₙ where m=1 to 10 and n=1 to 50. In a preferred aspect, the particulate benefit agent binding block is represented by the present formulas when m=1 and n=1 to 50, preferably 1 to 25, more preferably 1 to 10, and most preferably 1 to 3. In one embodiment, X1 or X2 may not be present (i.e., m=0) such that the particulate agent binding block may comprise of one or more of blocks of (X1-Y)ₙ or (Y—X2)ₙ, where n=1 to 50.

In one aspect, the two blocks X1 and X2 are comprised of positively charged amino acids when the target surface has a net negative charge. In another aspect, both X1 and X2 are comprises of negatively charged amino acids when the target surface has a net positive charge. The hydrophobic “Y” consists of a stretch of 3 to 10 contiguous hydrophobic amino acids. In a preferred aspect, the hydrophobic Y block is flanked by charged blocks X1 and X2. In one embodiment, one of the terminal charged blocks X1 or X2 may be absent when m=0; thereby the hydrophobic Y block may be directly attached to an optional spacer or to one of the respective body surface-binding domains when the respective spacer is not present. The hydrophobic amino acids are those having a hydrophobicity parameter of 0.8 or greater (see Table 2). The hydrophobic amino acids are therefore, phenylalanine, isoleucine, leucine, valine, tryptophan and tyrosine. The exact composition of each repeating instance of hydrophobic block Y in any of the multi-block peptides may vary so long as each occurrence of Y is comprised of 3 to 10 contiguous hydrophobic amino acids having a hydrophobicity parameter of 0.8 or greater.

The charged amino acids may be consecutive or interspersed among non-charged amino acids. In one embodiment, the charged amino acids within X1 and/or X2 are separated by a non-charged amino acid, such as glycine or proline. Preferred charged amino acids are the positively charged lysine, histidine and arginine.

There is no preferred mechanism of association of the particulate benefit agent with the peptide component's particulate benefit agent-binding domain. It is contemplated within the concept of the invention that the particulate benefit agent-binding domain interacts with the particulate benefit agent through noncovalent bonds. Ionic bonds or weaker electrostatic interactions are envisioned as contributing to the formation of an effective particulate benefit agent delivery adduct. In some embodiments of the peptide-particulate benefit agent adduct the particulate benefit agent may interact hydrophobically through the hydrophobic block of amino acids, Y.

It is further envisioned that the strength of the association between the peptide component and particulate benefit agent may be modulated by modifications of the surface of the particulate benefit agent. For example, various polymeric coatings discussed above may be applied to particulate benefit agent to impart a desired feature upon it, e.g., to render the particulate benefit agent particle more hydrophobic, or more negatively charged, or more stable in terms of the particles zeta potential, increased chemical stability in a particular medium, and the like.

### The Peptide-Particulate Benefit Agent Adduct

Stably-dispersed adducts are prepared by contacting at least one of the present multi-block peptides having the general structures defined herein with a particulate benefit agent for a period of time sufficient to form a stable adduct in solution. As defined herein, a stable adduct is defined as a peptide-particulate benefit agent adduct if the average particle size (e.g., as measured by D₅₀) does not increase by more than 50% over 7 days of storage under typical storage conditions. In one embodiment, the typical storage conditions comprises storage at room temperature (˜21° C.) in pH 5, 10 mM MES buffer (after washing to remove unbound or excess peptide).

The adduct zeta potential is less than or equal to −20 mV or greater than or equal to +20 mV or more (i.e., “±20 my”); more preferably at least ±30 mV and even more preferably at least ±40 mV. In a preferred embodiment, the adduct zeta potential is at least 20 mV, preferably at least 30 mV, and even more preferably at least 40 mV.

The average particle size of the peptide-particulate benefit agent adduct may vary. In one embodiment, the peptide-particulate benefit agent adduct ranges from 10 nm to 75 μm, preferably 10 nm to 10 μm, more preferably 100 nm to 5 μm, and most preferably 100 nm to 1 μm; further comprising a zeta potential as describe above. The average particle size may be determined using a light scattering method (such as dynamic light scattering) and may be reported in terms of a D₅₀ value.

The peptide-particulate benefit agent adducts may be contacted with a target body surface (i.e., the multi-block portion of the adduct comprises at least 2 body surface binding domains (“A1” and “A2”,) having affinity for the target body surface) under suitable conditions whereby the adduct binds non-covalently to the target body surface. In one embodiment, the presence of the multi-block proteins defined herein in the peptide-particulate benefit agent adduct increases the durability of the benefit agent for the body surface. In a preferred embodiment, the relative increase in durability is measured by washing the body surface comprising the bound adduct with a surfactant whereby the presence of the multi-block peptide defined herein increases the binding durability of the adduct when compared to a peptide-particulate benefit agent adduct lacking a peptide having the general structures defined herein. In a further embodiment, the surfactant used to measure for the relative increase in durability is solution of 2 wt % SLES. In a preferred embodiment, the increase in durability may be determined using a wash procedure as generally defined in Example 3.

### Recombinant Microbial Expression

The genes and gene products of the instant sequences may be produced in heterologous host cells, particularly in the cells of microbial hosts. Preferred heterologous host cells for expression of the instant genes and nucleic acid molecules are microbial hosts that can be found within the fungal or bacterial families and which grow over a wide range of temperature, pH values, and solvent tolerances. For example, it is contemplated that any of bacteria, yeast, and filamentous fungi may suitably host the expression of the present nucleic acid molecules. The polypeptides/proteins may be expressed intracellularly, extracellularly, or a combination of both intracellularly and extracellularly, where extracellular expression renders recovery of the desired polypeptides/protein from a fermentation product more facile than methods for recovery of protein produced by intracellular expression. Transcription, translation and the protein biosynthetic apparatus remain invariant relative to the cellular feedstock used to generate cellular biomass; functional genes will be expressed regardless. Examples of host strains include, but are not limited to, bacterial, fungal or yeast species such as Aspergillus, Trichoderma, Saccharomyces, Pichia, Phaffia, Kluyveromyces, Candida, Hansenula, Yarrowia, Salmonella, Bacillus, Acinetobacter, Zymomonas, Agrobacterium, Erythrobacter, Chlorobium, Chromatium, Flavobacterium, Cytophaga, Rhodobacter, Rhodococcus, Streptomyces, Brevibacterium, Corynebacteria, Mycobacterium, Deinococcus, Escherichia, Erwinia, Pantoea, Pseudomonas, Sphingomonas, Methylomonas, Methylobacter, Methylococcus, Methylosinus, Methylomicrobium, Methylocystis, Alcaligenes, Synechocystis, Synechococcus, Anabaena, Thiobacillus, Methanobacterium, Klebsiella, and Myxococcus. In one embodiment, bacterial host strains include Escherichia, Bacillus, and Pseudomonas. In a preferred embodiment, the bacterial host cell is Escherichia coli.

### Industrial Production

A variety of culture methodologies may be applied to produce the present polypeptides/proteins. Large-scale production of a specific gene product over expressed from a recombinant microbial host may be produced by batch, fed-batch or continuous culture methodologies. Batch and fed-batch culturing methods are common and well known in the art and examples may be found in Thomas D. Brock in Biotechnology: A Textbook of Industrial Microbiology, Second Edition, Sinauer Associates, Inc., Sunderland, Mass. (1989) and Deshpande, Mukund V., Appl. Biochem. Biotechnol., (1992) 36(3):227-234.

In one embodiment, commercial production is accomplished with a continuous culture. Continuous cultures are an open system where a defined culture media is added continuously to a bioreactor and an equal amount of conditioned media is removed simultaneously for processing. Continuous cultures generally maintain the cells at a constant high liquid phase density where cells are primarily in log phase growth. Alternatively, continuous culture may be practiced with immobilized cells where carbon and nutrients are continuously added and valuable products, by-products or waste products are continuously removed from the cell mass. Cell immobilization may be performed using a wide range of solid supports composed of natural and/or synthetic materials.

Recovery of the desired polypeptides/proteins from a batch or fed-batch fermentation, or continuous culture may be accomplished by any of the methods that are known to those skilled in the art. For example, when the polypeptide/protein is produced intracellularly, the cell paste is separated from the culture medium by centrifugation or membrane filtration, optionally washed with water or an aqueous buffer at a desired pH, then a suspension of the cell paste in an aqueous buffer at a desired pH is homogenized to produce a cell extract containing the desired peptidic product.

### Transformation and Expression

Construction of genetic cassettes and vectors that may be transformed in to an appropriate expression host is common and well known in the art. Typically, the vector or cassette contains sequences directing transcription and translation of the relevant chimeric construct, a selectable marker, and sequences allowing autonomous replication or chromosomal integration. Suitable vectors comprise a region 5′ of the gene which harbors transcriptional initiation controls and a region 3′ of the DNA fragment which controls transcriptional termination. It is most preferred when both control regions are derived from genes homologous to the transformed host cell, although it is to be understood that such control regions need not be derived from the genes native to the specific species chosen as a production host.

Transcription initiation control regions or promoters, which are useful to drive expression of the genetic constructs encoding the fusion peptides in the desired host cell are numerous and familiar to those skilled in the art. Virtually any promoter capable of driving these constructs is suitable for the present invention including, but not limited to CYC1, HIS3, GAL1, GAL10, ADH1, PGK, PHO5, GAPDH, ADC1, TRP1, URA3, LEU2, ENO, TPI (useful for expression in Saccharomyces); AOX1 (useful for expression in Pichia); and lac, ara (pBAD), tet, trp, IP_{L}, IP_{R}, T7, tac, and trc (useful for expression in Escherichia coli) as well as the amy, apr, npr promoters and various phage promoters useful for expression in Bacillus.

Termination control regions may also be derived from various genes native to the preferred hosts. Optionally, a termination site may be unnecessary; however, it is most preferred if included.

Preferred host cells for expression of the present polypeptides/proteins are microbial hosts that can be found broadly within the fungal or bacterial families and which grow over a wide range of temperature, pH values, and solvent tolerances. Because the transcription, translation, and the protein biosynthetic apparatus is the same irrespective of the cellular feedstock, genes are expressed irrespective of the carbon feedstock used to generate the cellular biomass. Large-scale microbial growth and functional gene expression may utilize a wide range of simple or complex carbohydrates, organic acids and alcohols, saturated hydrocarbons such as methane or carbon dioxide in the case of photosynthetic or chemoautotrophic hosts. The functional genes may be regulated, repressed or depressed by specific growth conditions, which may include the form and amount of nitrogen, phosphorous, sulfur, oxygen, carbon or any trace micronutrient including small inorganic ions. In addition, the regulation of functional genes may be achieved by the presence or absence of specific regulatory molecules that are added to the culture and are not typically considered nutrient or energy sources. Growth rate may also be an important regulatory factor in gene expression. Examples of host strains include, but are not limited to, fungal or yeast species such as Aspergillus, Trichoderma, Saccharomyces, Pichia, Yarrowia, Candida, Hansenula, or bacterial species such as Salmonella, Bacillus, Acinetobacter, Zymomonas, Agrobacterium, Erythrobacter, Chlorobium, Chromatium, Flavobacterium, Cytophaga, Rhodobacter, Rhodococcus, Streptomyces, Brevibacterium, Corynebacteria, Mycobacterium, Deinococcus, Escherichia, Erwinia, Pantoea, Pseudomonas, Sphingomonas, Methylomonas, Methylobacter, Methylococcus, Methylosinus, Methylomicrobium, Methylocystis, Alcaligenes, Synechocystis, Synechococcus, Anabaena, Thiobacillus, Methanobacterium, Klebsiella, and Myxococcus.

Preferred bacterial host strains include Escherichia, Pseudomonas, and Bacillus. In a highly preferred aspect, the bacterial host strain is Escherichia coli.

### Fermentation Media

Fermentation media in the present invention must contain suitable carbon substrates. Suitable substrates may include, but are not limited to, monosaccharides such as glucose and fructose, disaccharides such as lactose or sucrose, polysaccharides such as starch or cellulose or mixtures thereof and unpurified mixtures from renewable feedstocks such as cheese whey permeate, cornsteep liquor, sugar beet molasses, and barley malt. It is contemplated that the source of carbon utilized may encompass a wide variety of carbon containing substrates and will only be limited by the choice of organism. Although it is contemplated that all of the above mentioned carbon substrates and mixtures thereof are suitable in the present invention, preferred carbon substrates are glucose, fructose, and sucrose.

In addition to an appropriate carbon source, fermentation media must contain suitable minerals, salts, cofactors, buffers and other components, known to those skilled in the art, suitable for the growth of the cultures and promotion of the expression of the fusion peptides.

### Culture Conditions

Suitable culture conditions can be selected dependent upon the chosen production host. Typically, cells are grown at a temperature in the range of about 25° C. to about 40° C. in an appropriate medium. Suitable growth media may include common, commercially-prepared media such as Luria Bertani (LB) broth, Sabouraud Dextrose (SD) broth or Yeast Medium (YM) broth. Other defined or synthetic growth media may also be used and the appropriate medium for growth of the particular microorganism will be known by one skilled in the art of microbiology or fermentation science. The use of agents known to modulate catabolite repression directly or indirectly, e.g., cyclic adenosine 2′:3′-monophosphate, may also be incorporated into the fermentation medium. Suitable pH ranges for the fermentation are typically between pH 5.0 to pH 9.0, where pH 6.0 to pH 8.0 is preferred. Fermentations may be performed under aerobic or anaerobic conditions, where aerobic conditions are generally preferred.

### EXAMPLES

### General Methods

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions.

Standard recombinant DNA and molecular cloning techniques used herein are well known in the art and are described by Sambrook, J. and Russell, D., Molecular Cloning: A Laboratory Manual, Third Edition, Cold Spring Harbor Laboratory Press, Cold Spring Harbor, N.Y. (2001); and by Silhavy, T. J., Bennan, M. L. and Enquist, L. W., Experiments with Gene Fusions, Cold Spring Harbor Laboratory Cold Press Spring Harbor, N.Y. (1984); and by Ausubel, F. M. et. al., Short Protocols in Molecular Biology, 5^{th} Ed. Current Protocols and John Wiley and Sons, Inc., N.Y., 2002.

Materials and methods suitable for the maintenance and growth of bacterial cultures are also well known in the art. Techniques suitable for use in the following Examples may be found in Manual of Methods for General Bacteriology, Phillipp Gerhardt, R. G. E. Murray, Ralph N. Costilow, Eugene W. Nester, Willis A. Wood, Noel R. Krieg and G. Briggs Phillips, eds., American Society for Microbiology, Washington, D.C., 1994, or in Brock (supra). All reagents, restriction enzymes and materials used for the growth and maintenance of bacterial cells were obtained from BD Diagnostic Systems (Sparks, Md.), Invitrogen (Carlsbad, Calif.), Life Technologies (Rockville, Md.), QIAGEN (Valencia, Calif.) or Sigma-Aldrich Chemical Company (St. Louis, Mo.), unless otherwise specified.

The meaning of abbreviations used is as follows: “sec” means second(s), “min” means minute(s), “h” or “hr” means hour(s), “μL” means microliter(s), “mL” means milliliter(s), “L” means liter(s), “nm” means nanometer(s), “mm” means millimeter(s), “cm” means centimeter(s), “μm” means micrometer(s), “mM” means millimolar, “μM” means micromolar, “M” means molar, “mmol” means millimole(s), “μmole” means micromole(s), “g” means gram(s), “μg” means microgram(s), “mg” means milligram(s), “g” means the gravitation constant, “RCF” means relative centrifugal force, “rpm” means revolution(s) per minute, and “pfu” means plaque forming unit(s).

### Example 1

### Sodium Lauryl Ether Sulfate (SLES) Retention Assay on a Silica HPLC column

A series of silica-binding peptides (shown in Table 1) with different length, charge, and hydrophobicity index was tested for sodium lauryl ether sulfate (SLES) wash-resistance using a two-step protocol: 1) peptide loading onto a silica column followed by SLES elution, and 2) column regeneration. In the sample loading and SLES elution step, 25 μL of 0.5 mg/mL peptide in pH 5, 25 mM acetate buffer was injected into a silica HPLC column (Sepax Technologies, Inc., Newark, Del.; HP-Silica 2.1×150 mm column, Part #: 117005-2115) using a Model 2695 Separations Module (Waters, Inc., Milford, Mass.). The flow rate for all the mobile phases for HPLC was 0.5 mL/min. The same pH 5, 25 mM acetate buffer mobile phase was held constant for 10 min after sample injection to enable peptide binding to the silica column. Then, the mobile phase was linearly adjusted up to 0.3% SLES/pH 5, 25 mM acetate buffer within 3 min, followed by 1 hour isocratic elution at 0.3% SLES/pH 5, 25 mM acetate buffer. The mobile phase was then switched to deionized water, which ran for 10 minutes. Peptide fractions eluted in step 1 were counted as “buffer washout” or “SLES washout” if the elution peak occurred in the buffer loading/elution phase or the SLES elution phase, respectively. In step 2, the column regeneration step, the column was washed with regeneration buffer (1.5 M NaClO₄ in 70% acetonitrile, pH 3) for 20 min followed by deionized water wash and column equilibrium with pH 5, 25 mM acetate buffer. Peptide fractions eluted in this step were considered “SLES-resistant”. In the whole process, the column temperature was maintained at 25° C. The percentages of injected peptide counted as buffer washout, SLES washout, and SLES-resistant were calculated for each peptide based on elution peak areas, and are summarized in Table 1.

The isoelectric point for each peptide was calculated using European Molecular Biology Open Software Suite (EMBOSS) online software (Rice, P. et al., Trends in Genetics (2000) 16(6) pp. 276-277). The hydrophobicity index for each peptide was its average amino acid hydrophobicity parameter (the sum of the hydrophobicity parameter of each amino acid in the sequence divided by the number of amino acids in the sequence). The hydrophobicity parameters for the twenty canonical amino acids used here were developed by Black and Mould (Analytical Biochemistry, (1991) vol. 193, pp. 72-82) and are listed in Table 2. One-way analysis-of-variance (ANOVA) of the SLES-resistant fraction of each peptide, as determined by the HPLC analysis described above (Table 1), versus the maximum hydrophobic block length, indicated that SLES resistance increased dramatically when blocks of 3 or more consecutive hydrophobic amino acids were present in the sequence, as shown in Table 3. The ANOVA analysis deemed that >99.9% of the variation in % SLES resistance could be explained by variation in the maximum consecutive hydrophobic amino acid block size (p=0.000). The hydrophobicity parameters of the amino acids are listed in Table 2. The amino acids defined as “hydrophobic” for the purpose of this analysis are F, L, Y, I, V and W, all of which have a hydrophobicity parameter >0.8.

TABLE 1
Peptide sequences, characteristics and % SLES-resistant from silica
column HPLC retention assay.

| | Amino Acid | Number | | | Maximum | |
|---|---|---|---|---|---|---|
| Peptide | Sequence | of Amino | Isoelectric | Hydropho- | Hydrophobic | % SLES- |
| Name | (SEQ ID NO:) | Acids | Point | bicity Index | AA block size | resistant |
| Soti11 | HTNDNGQSTPRRD | 21 | 11.1 | 0.346 | 1 | 4.2 |
| | PPAFQRKK | | | | | |
| | (SEQ ID NO: 139) | | | | | |
| Soti23 | YKHERHYSQPLKVR | 16 | 10.4 | 0.39 | 1 | 4.7 |
| | HK | | | | | |
| | (SEQ ID NO: 140) | | | | | |
| HCF037 | HPPMNASHPHMH | 12 | 7.7 | 0.457 | 0 | 5.1 |
| | (SEQ ID NO: 141) | | | | | |
| CA4-13 | SDETGPQIPHRRAT | 16 | 9.3 | 0.399 | 1 | 5.3 |
| | WK | | | | | |
| | (SEQ ID NO: 142) | | | | | |
| HCF042 | HKLPSASRHHFH | 12 | 11.2 | 0.411 | 1 | 6.2 |
| | (SEQ ID NO: 143) | | | | | |
| A09 | IPWWNIRAPLNAK | 13 | 11.2 | 0.615 | 2 | 6.6 |
| | (SEQ ID NO: 144) | | | | | |
| IB5 | TPPELLHGDPRSK | 13 | 7.3 | 0.45 | 2 | 6.7 |
| | (SEQ ID NO: 145) | | | | | |
| HCF033 | HNYHYPHTGHMAH | 15 | 7.7 | 0.454 | 1 | 7.4 |
| | SA | | | | | |
| | (SEQ ID NO: 146) | | | | | |
| HCF032 | HNNHYPHGGHMAH | 15 | 7.7 | 0.432 | 1 | 7.8 |
| | AA | | | | | |
| | (SEQ ID NO: 147) | | | | | |
| HCF023 | HQNHQNHQNHQNH | 15 | 7.7 | 0.217 | 0 | 8.0 |
| | QN | | | | | |
| | (SEQ ID NO: 148) | | | | | |
| HP2 | AQSQLPDKHSGLHE | 20 | 9.0 | 0.404 | 1 | 8.1 |
| | RAPQRY | | | | | |
| | (SEQ ID NO: 149) | | | | | |
| HCF025 | HQLHQLHQLHQLH | 15 | 7.7 | 0.453 | 1 | 8.1 |
| | QL | | | | | |
| | (SEQ ID NO: 150) | | | | | |
| Soti8 | HHDRAEPRGMAAT | 19 | 9.3 | 0.428 | 1 | 8.2 |
| | LAQTIK | | | | | |
| | (SEQ ID NO: 151) | | | | | |
| HCF038 | HTKHSHTSPPPL | 12 | 9.3 | 0.456 | 1 | 8.4 |
| | (SEQ ID NO: 152) | | | | | |
| Soti13 | LNSMSDKHHGHQN | 21 | 10.4 | 0.328 | 1 | 8.5 |
| | TATRNQHK | | | | | |
| | (SEQ ID NO: 153) | | | | | |
| HCF036 | FASAHHTHTHHGAG | 15 | 7.7 | 0.462 | 1 | 8.6 |
| | F | | | | | |
| | (SEQ ID NO: 154) | | | | | |
| HCF027 | HSLHSLHSLHSLHS | 15 | 7.7 | 0.489 | 1 | 8.7 |
| | L | | | | | |
| | (SEQ ID NO: 155) | | | | | |
| HCF028 | HGLHGLHGLHGLH | 15 | 7.7 | 0.536 | 1 | 8.9 |
| | GL | | | | | |
| | (SEQ ID NO: 156) | | | | | |
| HCF039 | HVSHFHASRHER | 12 | 10.0 | 0.322 | 1 | 9.1 |
| | (SEQ ID NO: 157) | | | | | |
| HCF041 | HVSHHATGHTHT | 12 | 7.7 | 0.373 | 1 | 9.8 |
| | (SEQ ID NO: 158) | | | | | |
| HCF034 | HGHGHGAGAAHGH | 15 | 7.8 | 0.39 | 0 | 9.9 |
| | GH | | | | | |
| | (SEQ ID NO: 159) | | | | | |
| (EEAAK)₄ | GPEEAAKKEEAAKK | 29 | 8.8 | 0.357 | 0 | 10.1 |
| | EEAAKKEEAAKKPA | | | | | |
| | K | | | | | |
| | (SEQ ID NO: 160) | | | | | |
| HCF024 | HQAHQAHQAHQAH | 15 | 7.7 | 0.344 | 0 | 10.2 |
| | QA | | | | | |
| | (SEQ ID NO: 161) | | | | | |
| HCP5 | HHGTHHNATKQKN | 15 | 10.4 | 0.33 | 1 | 10.7 |
| | HV | | | | | |
| | (SEQ ID NO: 162) | | | | | |
| Soti5 | DHNNRQHAVEVRE | 21 | 10.4 | 0.282 | 1 | 11.0 |
| | NKTHTARK | | | | | |
| | (SEQ ID NO: 163) | | | | | |
| HCF040 | HGSKANHPHIRA | 12 | 11.2 | 0.397 | 1 | 12.0 |
| | (SEQ ID NO: 164) | | | | | |
| MEA4 | HINKTNPHQGNHHS | 20 | 10.4 | 0.307 | 1 | 13.4 |
| | EKTQRQ | | | | | |
| | (SEQ ID NO: 165) | | | | | |
| HCF035 | AHHASTGGTSSAHH | 15 | 7.7 | 0.407 | 0 | 13.7 |
| | A | | | | | |
| | (SEQ ID NO: 166) | | | | | |
| HCP5 | HHGTHHNATKQKN | 39 | 10.9 | 0.368 | 1 | 15.3 |
| dimer | HVGGSGPGSGGHH | | | | | |
| | GTHHNATKQKNHV | | | | | |
| | (SEQ ID NO: 167) | | | | | |
| HCF029 | GKGKGKGKGKGKG | 15 | 11.2 | 0.399 | 0 | 16.4 |
| | KG | | | | | |
| | (SEQ ID NO: 168) | | | | | |
| HCF026 | HSGHSGHSGHSGH | 15 | 7.7 | 0.342 | 0 | 16.8 |
| | SG | | | | | |
| | (SEQ ID NO: 169) | | | | | |
| Gray3 | HDHKNQKETHQRH | 16 | 10.1 | 0.25 | 0 | 17.9 |
| | AAK | | | | | |
| | (SEQ ID NO: 170) | | | | | |
| HCF031 | AVAGKGKGKGKGA | 15 | 10.9 | 0.517 | 1 | 20.6 |
| | VA | | | | | |
| | (SEQ ID NO: 171) | | | | | |
| HCF030 | AKAKPAKAKPAKAK | 15 | 11.1 | 0.495 | 0 | 21.1 |
| | A | | | | | |
| | (SEQ ID NO: 172) | | | | | |
| CXHG1 | PWRRRIVWRFMRN | 28 | 12.6 | 0.554 | 3 | 52.7 |
| | HALASMLWLSVSTV | | | | | |
| | K | | | | | |
| | (SEQ ID NO: 173) | | | | | |
| OP-1 | RKKRKKFYFYFY | 12 | 10.6 | 0.564 | 6 | 86.8 |
| | (SEQ ID NO: 174) | | | | | |
| TonB- | GPEPEPEPEPIPEP | 38 | 8.8 | 0.506 | 3 | 100.0 |
| K(Biotin) | PKEAPVVIEKPKPKP | | | | | |
| | KPKPKPPAK-(Biotin) | | | | | |
| | (SEQ ID NO: 175) | | | | | |
| TonBHis | GPEPEPEPEPIPEP | 43 | 7.7 | 0.463 | 3 | 100.0 |
| | PKEAPVVIEKPKPKP | | | | | |
| | KPKPKPPAHHHHHH | | | | | |
| | (SEQ ID NO: 176) | | | | | |

TABLE 2
Hydrophobicity parameters of the twenty canonical amino acids.

| Amino acid | Amino acid code | | Hydrophobicity |
|---|---|---|---|
| name | 3-Letter | 1-Letter | parameter |
| Alanine | Ala | A | 0.616 |
| Cysteine | Cys | C | 0.68 |
| Aspartate | Asp | D | 0.028 |
| Glutamate | Glu | E | 0.043 |
| Phenylalanine | Phe | F | 1 |
| Glycine | Gly | G | 0.501 |
| Histidine | His | H | 0.165 |
| Isoleucine | Ile | I | 0.943 |
| Lysine | Lys | K | 0.283 |
| Leucine | Leu | L | 0.943 |
| Methionine | Met | M | 0.738 |
| Asparagine | Asn | N | 0.236 |
| Proline | Pro | P | 0.711 |
| Glutamine | Gln | Q | 0.251 |
| Arginine | Arg | R | 0 |
| Serine | Ser | S | 0.359 |
| Threonine | Thr | T | 0.45 |
| Valine | Val | V | 0.825 |
| Tryptophan | Trp | W | 0.878 |
| Tyrosine | Tyr | Y | 0.88 |

TABLE 3
Results breakdown for the SLES-resistant percentage of each peptide
versus the maximal block length of consecutive hydrophobic
amino acids in each peptide's sequence.

| Maximum | | | |
|---|---|---|---|
| Hydrophobic | Number of | Mean % SLES | Standard Deviation of |
| Block Length | Peptides | Resistant | % SLES Resistant |
| 0 | 10 | 12.93 | 5.04 |
| 1 | 22 | 9.33 | 3.64 |
| 2 | 2 | 6.65 | 0.13 |
| 3 | 3 | 84.23 | 27.32 |
| 6 | 1 | 86.82 | — |

### Example 2

### Multi-block Peptides with a Rationally-designed Pigment-binding Domain for Stable Peptide-pigment Adducts

Based on the results of the SLES retention assay shown in Example 1, peptide domains intended for binding to silica-coated particulate pigments were designed with positively-charged blocks and ≧3-mer hydrophobic amino acid blocks. These blocks such as (PK)₆APWI(PK)₆—(PK)₆APWI(PK)₆ (SEQ ID NO: 198) and (GK)₆APWI(GK)₆-(GK)₆APWI(GK)₆ (SEQ ID NO: 199) are exemplified in complete multi-block peptide components such as SEQ ID NO: 177 and SEQ ID NO: 181, respectively (See Table 4). The rationally-designed pigment binding domains (SEQ ID NOs: 198 and 199) were flanked with hair-binding domains HP2 (SEQ ID NO: 149), Gray3 (SEQ ID NO: 170), and MEA4 (SEQ ID NO: 165), with or without spacer peptides, to form multi-block peptides as provided in Table 4 (SEQ ID NOs: 177-182). The peptides were recombinantly produced by E. coli expression using techniques well-known to those of ordinary skill in the art.

Solutions of peptide dissolved in water were added to 0.25% silica-coated red iron oxide dispersion in pH 7.5, 25 mM Tris to a final concentration of 10 μM peptide. The silica-coated red iron oxide pigments were made according to the methods disclosed in U.S. Pat. No. 2,885,366, incorporated herein by reference. The peptide-pigment mixtures were vortexed at medium intensity for 10 min to ensure peptide-pigment association followed by centrifugation at 9300 RCF (approximately 10,000 rpm) to remove excess unbound peptide, if any. Then the centrifuged peptide-pigment adduct pellets were dispersed into the same volume of pH 5, 10 mM MES buffer by vortexing. The goal was to obtain a higher degree of stability for the formed peptide-pigment adducts due to the higher degree of ionization of cationic amino acids such as lysine in the peptide sequences at pH 5, compared with pH 7.5. Then, the particle size distribution and zeta potential of the adduct dispersions were measured using a Malvern Mastersizer 2000 and a Malvern Zetasizer Nano-ZS, respectively (Malvern Instruments Ltd, Malvern, UK). The adduct zeta potentials at day 0 and the volume-based median particle diameters (D₅₀) at days 0 and 7 are summarized in Table 4. The adduct particle size was regarded as stable if its D₅₀ increased less than 50% over 7 days of storage. Five of the six peptide-pigment adducts shown in Table 4 were stable by this definition, with the HC913 (SEQ ID NO: 181) adduct missing the mark only slightly (64% increase). Peptides HC907 (SEQ ID NO: 177) and HC908 (SEQ ID NO: 178), as well as HC913 (SEQ ID NO: 181) and HC915 (SEQ ID NO: 182), represent two pairs of peptides with identical A1 and A2 binding domains and identical (X1-Y—X2) segments for pigment association, but with the insertion of spacer sequences in HC908 (SEQ ID NO: 178) and HC915 (SEQ ID NO: 182). These latter two peptides gave better adduct size stability than their respective counterparts lacking the spacer sequences, HC907 (SEQ ID NO: 177) and HC913 (SEQ ID NO: 181). On the other hand, for HC910 (SEQ ID NO: 179) and HC912 (SEQ ID NO: 180), another pair of peptides with identical A1 and A2 binding domains and identical (X1-Y—X2) segments for pigment association, but with additional spacer sequences in HC912, adducts made with these peptides exhibited approximately equally good size stability over 7 days.

TABLE 4
Multi-block peptide sequences and measurements of their pigment
adduct particle size and zeta potential.

| | | | | Adduct | Adduct | |
|---|---|---|---|---|---|---|
| | | | Adduct Zeta | D₅₀, | D₅₀, | % change in |
| SEQ ID | Peptide | | potential, | Initial | Day 7 | Adduct D₅₀ |
| NO: | Name | Sequence Description¹ | Initial (mV) | (nm) | (nm) | after 7 days |
| 177 | HC907 | PS-HP2- | 32 | 344 | 442 | 28 |
| | | PKPKPKPKPKPKAPVVI | | | | |
| | | PKPKPKPKPKPKPKPK | | | | |
| | | PKPKPKPKAPVVIPKPK | | | | |
| | | PKPKPKPK-Gray3 | | | | |
| 178 | HC908 | PS-HP2- | 26 | 309 | 329 | 6 |
| | | GSSGPGSGSPKPKPK | | | | |
| | | PKPKPKAPVVIPKPKPK | | | | |
| | | PKPKPKPKPKPKPKPK | | | | |
| | | PKAPVVIPKPKPKPKPK | | | | |
| | | PKGSSGPGSGS-Gray3 | | | | |
| 179 | HC910 | PS-HP2- | 34 | 305 | 301 | −1 |
| | | PKPKPKPKPKPKAPVVI | | | | |
| | | PKPKPKPKPKPKPKPK | | | | |
| | | PKPKPKPKAPVVIPKPK | | | | |
| | | PKPKPKPK-MEA4 | | | | |
| 180 | HC912 | PS-HP2- | 30 | 546 | 528 | −3 |
| | | GSSGPGSGSSGPGSG | | | | |
| | | SSGPKPKPKPKPKPKA | | | | |
| | | PVVIPKPKPKPKPKPKP | | | | |
| | | KPKPKPKPKPKAPVVIP | | | | |
| | | KPKPKPKPKPKGSSGP | | | | |
| | | GSGSSGPGSGSSG- | | | | |
| | | MEA4 | | | | |
| 181 | HC913 | PS-Gray3- | 34 | 414 | 678 | 64 |
| | | GKGKGKGKGKGKAPV | | | | |
| | | VIGKGKGKGKGKGKG | | | | |
| | | KGKGKGKGKGKAPVVI | | | | |
| | | GKGKGKGKGKGK- | | | | |
| | | MEA4 | | | | |
| 182 | HC915 | PS-Gray3- | 29 | 345 | 386 | 12 |
| | | GSSGPGSGSSGPGSG | | | | |
| | | SSGPGSGSSGGKGKG | | | | |
| | | KGKGKGKAPVVIGKGK | | | | |
| | | GKGKGKGKGKGKGKG | | | | |
| | | KGKGKAPVVIGKGKGK | | | | |
| | | GKGKGKGSSGPGSGS | | | | |
| | | SGPGSGSSGPGSGSS | | | | |
| | | GPGSSG-MEA4 | | | | |

¹= Biopanned peptide names in italics.

### Example 3

### Effect of the Lengths of the Charged Amino Acid Block and Hydrophobic Amino Acid Block in Pigment Binding

Multi-block peptides HC1035 (SEQ ID NO: 187), HC1036 (SEQ ID NO: 188), and HC1037 (SEQ ID NO: 189), consisting of hair-binding domains HP2 and MEA4 at either terminus, and a charged amino acid block with increasing length (6, 12 and 18 lysine residues) as the pigment binding-domain in between, were designed and produced to test the effect of charged amino acid block length on peptide-pigment adduct performance. With 18 or 24 total lysine residues in the pigment-binding domain, hydrophobic blocks from one to three amino acids in length were tested in multi-block peptides HC1041 (SEQ ID NO: 190), HC1042 (SEQ ID NO: 191), HC1044 (SEQ ID NO: 192), HC1047 (SEQ ID NO: 193), and HC1055 (SEQ ID NO: 194), to evaluate the effect of hydrophobic block length on peptide-pigment adduct performance. These peptides were used to form adducts with silica-coated red iron oxide pigments using the same procedure as described in Example 2: a peptide-in-water stock solution was added to a 0.25% silica-coated red iron oxide dispersion in pH 7.5, 25 mM Tris buffer to a final peptide concentration of 10 μM. The peptide-pigment mixtures were vortexed at medium intensity for 10 min to ensure peptide-pigment association. The mixtures were then centrifuged at 9300 RCF (approximately 10,000 rpm) to remove excess unbound peptide, if any. Then the centrifuged peptide-pigment adduct pellets were dispersed into the same volume of pH 5, 10 mM MES buffer with vortexing. The particle size distribution and zeta potential of the adduct dispersions were measured initially and after 7 days using a Malvern Mastersizer 2000 and a Malvern Zetasizer_Nano-ZS, respectively. The volume-based median particle diameters (D₅₀) and zeta potential for each adduct sample on day 0 and day 7 are shown in Table 5. Adducts were deemed stable if their D₅₀ increased less than 50% over 7 days of storage.

Regarding the adduct formation process, the zeta potential values of the adducts indicates that a peptide with only six charged amino acids in its pigment-binding domain (HC1035; SEQ ID NO: 187) was able to bring about a reversal of the sign of the zeta potential of the pigment particles, considering the no-peptide control adduct's zeta potential of −34 mV (Table 5). However, HC1035 did not form a well-dispersed adduct initially, as evidenced by its large particle size. Nonetheless, after 7 days, the zeta potential of the HC1035 adduct had grown from 10 to 20 mV and the adduct's mean particle size became smaller. It is likely that a longer time was required for HC1035 to optimize its conformation on the pigment surface and maximize the zeta potential of it's adduct. When the number of charged amino acids in the pigment binding domain increased to 12 or more, the adduct zeta potential leveled off at about 30 mV, and the formed adduct particles were small and stable during the test period. Thus, the length of the charged amino acid block in the peptide's pigment-binding domain greatly impacted the stability and zeta potential of the peptide-pigment adducts, as indicated in Table 5.

The adducts were directly applied to hair for hair coloring. For each adduct, two hair tresses were colored in the same way: 0.5 mL of adduct dispersion was transferred into a plastic weighing boat, and the 1 cm wide, 2.5 cm long natural white hair tress from International Hair Importers & Products, Inc. was added. The adduct dispersion was spread onto the hair tress using mild gloved-finger embrocation for 30 seconds on each side. After an additional 9 minutes of quiescent contact with the adduct, the hair tress was rinsed with tap water, blotted with paper towels, and air-dried. L*, a*, b* color measurements were taken for each hair sample to quantify initial color uptake. L*, a*, b* color measurements were also taken for untreated natural white hair as a reference for ΔE color difference calculations. AE was calculated in the standard way as ΔE=((L*−L*_{ref})²+(a*−a*_{ref})²+(b*−b*_{ref})²)^{0.5}; where L*_{ref}, a*_{ref}, and b*_{ref} are the initial reference readings and L*, a*, and b* are the subsequent readings.

After initial color uptake readings were taken, the hair tresses were subjected to 5 cycles of washing with 2% SLES. Each wash cycle was done with bead embrocation as follows: tresses were placed one-to-a-well in a 24-well plate along with beads (four 3-mm glass beads, two 4-mm glass beads, and two 6.35-mm glass beads per well). Each tress was wet with deionized water, excess water was then removed by suction, then 1 mL of 2% SLES was added. The plate containing the tresses, beads, and SLES solution was capped and then vortexed at high intensity for 30 seconds, after which suds were removed by suction. Four milliliters of deionized water was added to each well, swirled gently, and then aspirated out. The tresses were further rinsed individually with a jet of deionized water for 10-15 seconds on each side. Excess water was removed by blotting with paper towel. The tresses were then air-dried. The average values of ΔE for the duplicate sets of hair tresses after a number of bead embrocation cycles were measured for each peptide-pigment adduct sample. The peptide sequence details for each adduct, and the average ΔE values of the colored hair tresses initially and after one, two, and five cycles of bead embrocation with 2% SLES (ec1, ec2 and ec5) are presented in Table 6.

The length of the hydrophobic amino acid block in the peptide's pigment-binding domain impacted the adducts' color retention (SLES wash-resistance) on hair as shown in Table 6. With a maximum of 0-2 consecutive hydrophobic amino acids in the pigment-binding domain, typically ˜50% color retention was obtained after 5 bead embrocation cycles in 2% SLES, exemplified by the adducts formed with HC1035 (SEQ ID NO: 187), HC1036 (SEQ ID NO: 188), HC1037 (SEQ ID NO: 189), HC1041 (SEQ ID NO: 190), HC1042 (SEQ ID NO: 191), and HC1044 (SEQ ID NO: 192). With up to 3 consecutive hydrophobic amino acids in the pigment-binding domain, HC1047 (SEQ ID NO: 193) and HC1055 (SEQ ID NO: 194) formed adducts that gave ˜60% color retention after 5 bead embrocation cycles in 2% SLES, an improvement by a factor of ˜1.2. The apparently high color retention of the no-peptide control in Table 6 (55.8%) cannot be considered on-par with the other samples, as the initial color deposition was much lower with the no-peptide control.

In sum, the hair color retention studies demonstrate that using adducts with a pigment binding domain having at least three hydrophobic amino acids was unexpectedly found to increase the amount of color retained by shampooed hair. This increase was as much as 20% when compared to adducts formed with fewer hydrophobic amino acids.

TABLE 5
Multi-block peptide sequences and their pigment adduct particle size and zeta potential
stability measurements.

| | | | | | | | | % |
|---|---|---|---|---|---|---|---|---|
| | | # of | | | | | | change |
| | | charged | | Adduct | Adduct | | | in |
| | | AA in | | Zeta | Zeta | Adduct | Adduct | Adduct |
| | | pigment- | Max. | potential, | potential, | Z-avg, | Z-avg, | Z-avg |
| Peptide | Sequence Description¹ | binding | Hydrophobic. | Initial | Day 7 | Initial | Day 7 | after 7 |
| Name | (SEQ ID NO:) | domain | Block Length | (mV) | (mV) | (nm) | (nm) | days |
| no | — | — | — | −34 | −31 | 156 | 158 | 1 |
| peptide | | | | | | | | |
| HC1035 | PS-HP2-(PK)₆-MEA4 | 6 | 0 | 10 | 20 | 773 | 269 | −65 |
| | (SEQ ID NO: 187) | | | | | | | |
| HC1036 | PS-HP2-(PK)₁₂-MEA4 | 12 | 0 | 30 | 29 | 198 | 181 | −9 |
| | (SEQ ID NO: 188) | | | | | | | |
| HC1037 | PS-HP2-(PK)₁₈-MEA4 | 18 | 0 | 32 | 27 | 252 | 232 | −8 |
| | (SEQ ID NO: 189) | | | | | | | |
| HC1041 | PS-HP2-(PK)₆W(PK)₆V(PK)₆- | 18 | 1 | 31 | 30 | 206 | 206 | 0 |
| | MEA4 | | | | | | | |
| | (SEQ ID NO: 190) | | | | | | | |
| HC1042 | PS-HP2-(PK)₆W(PK)₁₂V(PK)₆- | 24 | 1 | 32 | 30 | 240 | 215 | −10 |
| | MEA4 | | | | | | | |
| | (SEQ ID NO: 191) | | | | | | | |
| HC1044 | PS-HP2-(PK)₆WW(PK)₆VW | 18 | 2 | 30 | 29 | 196 | 203 | 4 |
| | (PK)₆-MEA4 | | | | | | | |
| | (SEQ ID NO: 192) | | | | | | | |
| HC1047 | PS-HP2-(PK)₆VVI(PK)₆VVI | 24 | 3 | 30 | 30 | 191 | 183 | −4 |
| | (PK)₆VVI(PK)₆-MEA4 | | | | | | | |
| | (SEQ ID NO: 193) | | | | | | | |
| HC1055 | PS-HP2- | 24 | 3 | 31 | 33 | 195 | 189 | −3 |
| | GPTTTTSSKTTTTSSKPA- | | | | | | | |
| | (PK)₁₂VVI(PK)₆VVI(PK)₆- | | | | | | | |
| | GPTTTTSSKTTTTSSKPA-MEA4 | | | | | | | |
| | (SEQ ID NO: 194) | | | | | | | |

¹= Biopanned peptide names in italics.

TABLE 6
Color uptake and color retention for peptide-pigment adducts made with peptides with different
charge and hydrophobic block length in their pigment-binding domain.

| | | # of | | | | | | |
|---|---|---|---|---|---|---|---|---|
| | | charged | | | | | | % retention |
| | | AA in | Max. | | | | | of |
| | | pigment- | Hydropho- | Mean ΔE − | Mean | Mean | Mean | deposition |
| Peptide | Sequence Description¹ | binding | bic Block | color | ΔE − | ΔE − | ΔE − | ΔE after |
| Name | (SEQ ID NO: ) | domain | Length | deposition | ec1 | ec2 | ec5 | ec5 |
| no | — | — | — | 7.7 | 5.9 | 5.0 | 4.3 | 55.8 |
| peptide | | | | | | | | |
| HC1035 | PS-HP2-(PK)₆-MEA4 | 6 | 0 | 33.3 | 24.5 | 19.6 | 15.8 | 47.4 |
| | (SEQ ID NO: 187) | | | | | | | |
| HC1036 | PS-HP2-(PK)₁₂-MEA4 | 12 | 0 | 37.0 | 28.5 | 23.4 | 17.5 | 47.4 |
| | (SEQ ID NO: 188) | | | | | | | |
| HC1037 | PS-HP2-(PK)₁₈-MEA4 | 18 | 0 | 37.9 | 30.7 | 25.4 | 19.7 | 52.0 |
| | (SEQ ID NO: 189) | | | | | | | |
| HC1041 | PS-HP2-(PK)₆W(PK)₆V(PK)₆-MEA4 | 18 | 1 | 34.7 | 27.6 | 23.2 | 17.2 | 49.6 |
| | (SEQ ID NO: 190) | | | | | | | |
| HC1042 | PS-HP2-(PK)₆W(PK)₁₂V(PK)₆-MEA4 | 24 | 1 | 33.7 | 25.5 | 19.5 | 14.4 | 42.7 |
| | (SEQ ID NO: 191) | | | | | | | |
| HC1044 | PS-HP2-(PK)₆WW(PK)₆VW(PK)₆-MEA4 | 18 | 2 | 39.3 | 32.9 | 26.0 | 17.9 | 45.5 |
| | (SEQ ID NO: 192) | | | | | | | |
| HC1047 | PS-HP2-(PK)₆VVI(PK)₆VVI(PK)₆VVI | 24 | 3 | 39.4 | 34.6 | 30.9 | 24.5 | 62.1 |
| | (PK)₆-MEA4 | | | | | | | |
| | (SEQ ID NO: 193) | | | | | | | |
| HC1055 | PS-HP2-GPTTTTSSKTTTTSSKPA- | 24 | 3 | 39.3 | 33.0 | 30.0 | 23.3 | 59.4 |
| | (PK)₁₂VVI(PK)₆VVI(PK)₆- | | | | | | | |
| | GPTTTTSSKTTTTSSKPA-MEA4 | | | | | | | |
| | (SEQ ID NO: 194) | | | | | | | |

¹= Biopanned peptide names in italics.

### Example 4

### Hair Coloring and Retention Using Peptide-Pigment Adduct Dispersions Made with Peptides of Different Architectures

Multi-block peptides HC907 (SEQ ID NO: 177), HC908 (SEQ ID NO: 178), HC910 (SEQ ID NO: 179), and HC912 (SEQ ID NO: 180), consisting of a hair-binding domain at either end, a rationally-designed silica-binding sequence ((PK)₆APWI(PK)₆-(PK)₆APWI(PK)₆) (SEQ ID NO: 198) in the interior, and optional spacer peptide sequences at the domain junctions, were used to form adducts with silica-coated red iron oxide pigment. HC257 (SEQ ID NO: 183) and HC263 (SEQ ID NO: 184), with an overall neutrally-charged peptide sequence in place of rationally-designed silica-binding sequences, served as control peptides for HC907 (SEQ ID NO: 177) and HC908 (SEQ ID NO: 178), respectively. HC353 (SEQ ID NO: 185), with a hair-binding domain followed by a pigment-binding domain, and containing the HC263 (SEQ ID NO: 184) sequence (save for the 6-His tag) as the hair-binding domain, also served as a control peptide. HC360 (SEQ ID NO: 186), also with an overall neutrally-charged peptide sequence instead of rationally-designed silica-binding sequences, served as yet another control peptide. Peptide-pigment adducts were formed using the same procedure described in Example 2. The same procedure as described in Example 3 was used to color hair tresses with the adducts. Color deposition and color retention on the tresses after a number of bead embrocation cycles with 2% SLES were measured as in Example 3. These data are summarized in Table 7.

Compared to their respective control peptides with net-neutral amino acid sequences instead of charged sequences in between HBPs, hair tresses colored with adducts made from HC907 (SEQ ID NO: 177), HC908 (SEQ ID NO: 178) and HC910 (SEQ ID NO: 179) exhibited at least 5 ΔE units greater color retention after 5 embrocation cycles. Adducts of HC907, HC908 and HC910 also retained color on hair better than the HC353 (SEQ ID NO: 185) adduct. HC353 contains the sequence of HC263 (SEQ ID NO: 184) (save for the 6-His tag) as its hair-binding domain, has a long stretch of charged amino acids in its pigment-binding domain, and has the longest sequence of any peptide tested, but the color retention of its adduct was only on par with that of HC263. This suggests that in addition to charged and hydrophobic amino acid content, the architecture and distribution of charges and hydrophobic residues within multi-block peptides are also important for their performance. In this respect, sequences such as HC907 (SEQ ID NO: 177), 908 (SEQ ID NO: 178), 910 (SEQ ID NO: 179) and 912 (SEQ ID NO: 180), in which the hair-binding domains flank a rationally-designed pigment binding domain at either end, proved to have an edge in color retention over alternative architectures such as that of HC353 (SEQ ID NO: 185).

TABLE 7
Color uptake and color retention for peptide-pigment adducts.

| | | Mean ΔE − | | | | % retention of |
|---|---|---|---|---|---|---|
| Peptide | Sequence Description¹ | color | Mean ΔE − | Mean ΔE − | Mean ΔE − | deposition ΔE |
| Name | (SEQ ID NO: ) | deposition | ec1 | ec2 | ec5 | after ec5 |
| no peptide | — | 4.0 | — | — | — | — |
| HC257 | PS-HP2-GP(GAGGAGGSGGS)₂PA-Gray3- | 36.6 | 28.2 | 24.3 | 19.4 | 52.9 |
| | GSGGGGSP-HHHHHH | | | | | |
| | (SEQ ID NO: 183) | | | | | |
| HC263 | PS-HP2- | 35.4 | 29.9 | 25.5 | 19.1 | 53.9 |
| | GPEPEPEPEPIPEPPKEAPVVIEKPKPKPKPKPK | | | | | |
| | PPA-Gray3-GSGGGGSP-HHHHHH | | | | | |
| | (SEQ ID NO: 184) | | | | | |
| HC353 | PS-HP2- | 34.6 | 29.2 | 24.1 | 18.6 | 53.6 |
| | GPEPEPEPEPIPEPPKEAPVVIEKPKPKPKPKPK | | | | | |
| | PPA-Gray3-GSGGGGSP-Rfe1- | | | | | |
| | GKGKGKGKGKGKGKGK GKGKG-Rfe1-GK | | | | | |
| | (SEQ ID NO: 185) | | | | | |
| HC907 | PS-HP2-PKPKPKPKPKPKAPVVIPKPK | 39.4 | 34.8 | 32.2 | 26.5 | 67.2 |
| | PKPKPKPKPK | | | | | |
| | PKPKPKPKPKAPVVIPKPKPKPKPKPK-Gray3 | | | | | |
| | (SEQ ID NO: 177) | | | | | |
| HC908 | PS-HP2-GSSGPGSGSPKPKPKPKPKPKAPVVIPK | 39.5 | 34.8 | 32.1 | 24.5 | 61.9 |
| | PKPKPKPKPKPKPKPKPKPKPKAPVVIPKPKPKPKP | | | | | |
| | KPKGSSGPGSGS-Gray3 | | | | | |
| | (SEQ ID NO: 178) | | | | | |
| HC360 | PR-HP2-GPEPEPEPEPIPEPPK | 35.1 | 30.8 | 26.5 | 20.0 | 56.9 |
| | EAPVVIEKPKPKPKPKPKPPA-MEA4- | | | | | |
| | GSGGGGSPHHHHHH | | | | | |
| | (SEQ ID NO: 186) | | | | | |
| HC910 | PS-HP2-PKPKPKPKPKPKAPVVIPKPKPKPKPK | 38.8 | 35.1 | 31.0 | 25.3 | 65.2 |
| | PKPKPKPK PKPKPKAPVVIPKPKPKPKPKPK- | | | | | |
| | MEA4 | | | | | |
| | (SEQ ID NO: 179) | | | | | |
| HC912 | PS-HP2-GSSGPGSGSSGPGS | 40.3 | 35.4 | 29.5 | 23.7 | 58.8 |
| | GSSGPKPKPKPKPKPKAPVVIPKPKPKPK | | | | | |
| | PKPKPKPKPKPKPKPKAPVVIPKPKPKPKP | | | | | |
| | KPKGSSGPGSGSSGPGSGSSG-MEA4 | | | | | |
| | (SEQ ID NO: 180) | | | | | |

¹= Biopanned peptide names in italics.

### Example 5

### Hair Coloring with Peptide-Carbon Black Adducts

Jupiter carbon black dispersion (product code 57P30993, lot number M583226) was obtained from DuPont Digital Printing (E.I. duPont de Nemours and Company, Inc., Wilmington, Del.). HC907 (SEQ ID NO: 177) and HC910 (SEQ ID NO: 179) were used to form adducts with the Jupiter carbon black dispersion using the same procedure as described in Example 2.

The adducts were directly applied to hair for hair coloring. For each adduct, two hair tresses were colored in the same way, as described in Example 3. After initial color uptake readings, the hair tresses were subjected to 5 cycles of bead embrocation washing in 2% SLES using the same procedure described in Example 3. The initial color uptake (including the L*, a*, b* scores), and the ΔE values of color retained after the first, second and fifth SLES wash cycles are listed in Table 8. Compared to the peptide-free control, the use of peptides HC907 (SEQ ID NO: 195) and HC910 (SEQ ID NO: 197) enhanced the initial color uptake and color retained after SLES wash cycles, thus demonstrating the effectiveness of these peptides with a very different pigment than red iron oxide.

TABLE 8
Color uptake and color retention for peptide-carbon black adducts.

| | | | | Mean ΔE - | Mean | Mean | Mean |
|---|---|---|---|---|---|---|---|
| | | | | color | ΔE - | ΔE - | ΔE - |
| Adduct/hair sample | L* | a* | b* | deposition | ec1 | ec2 | ec5 |
| Natural White Hair | 72.3 | 3.8 | 27.3 | — | — | — | — |
| Reference | | | | | | | |
| Jupiter Carbon | 52.2 | 0.7 | 11.8 | 25.6 | 21.2 | 20.2 | 18.1 |
| Black only | | | | | | | |
| (peptide-free | | | | | | | |
| control) | | | | | | | |
| HC907/Jupiter | 46.5 | 0.3 | 9.6 | 31.5 | 27.4 | 25.4 | 21.8 |
| Carbon Black | | | | | | | |
| adduct | | | | | | | |
| HC910/Jupiter | 41.3 | 0.4 | 7.8 | 36.8 | 32.0 | 29.5 | 25.9 |
| Carbon Black | | | | | | | |
| adduct | | | | | | | |

### Example 6

### Use of a Mixture of Red and Black Pigment-peptide Adducts to Achieve Brown Color on Hair

HC910 (SEQ ID NO: 179) was used to form adducts with silica-coated red iron oxide pigment and, separately, with silica-coated black iron oxide pigment using the same procedure described in Example 2, except the black iron oxide dispersion was 0.5% solids while the red iron oxide dispersion was 0.25% solids. The two peptide-pigment adduct dispersions were then mixed at different red/black volume ratios ranging from 2 to 10. Each adduct mixture was applied to two hair tresses using the following procedure: 0.5 mL of an adduct mixture was transferred into a plastic weighing boat, and a 1-cm wide by 2-cm long natural white hair tress from International Hair Importers & Products, Inc. was added. The adduct mixture was spread onto the hair tress using mild gloved-finger embrocation for 30 seconds on each side. After one more minute of quiescent contact with the adduct mixture, the hair tress was rinsed with tap water, blotted with paper towels, and air-dried. For adduct mixtures with a red/black ratio of 2, a five-inch long hair tress was also used to demonstrate the brown coloring using the same procedure as described above, but using 2.5 mL of adduct mixture. L*, a*, b* color values were measured for each hair sample. L*, a*, b* reference values for untreated natural white hair were also measured for use in ΔE color difference calculations, ≢E =((L*−L*_{ref})²+(a*−a*_{ref})²+(b*−b*_{ref})²)^{0.5}. The L*, a*, b* scores and the calculated ΔE color difference values are summarized in Table 9. With increasing black adduct content (decreasing red/black ratios), lower values of L*, a*, and b* were generally obtained, and the hair samples turned from red to brown.

TABLE 9
L*, a*, b* color scores and ΔE color difference values
for hair samples colored by red/black adduct mixture

| | red adduct/ | | | | | |
|---|---|---|---|---|---|---|
| | black adduct | | | | | Visual |
| Sample | volume ratio | L* | a* | b* | ΔE | color |
| natural white | | 73.9 | 3.2 | 24.5 | — | yellow- |
| hair reference | | | | | | white |
| D100751-177-3A | 10 | 48.9 | 20.6 | 22.2 | 57.6 | red |
| D100751-177-3B | 10 | 45.2 | 21.9 | 22.5 | 55.0 | red |
| D100751-177-4A | 8 | 46.8 | 20.6 | 22.0 | 55.7 | red |
| D100751-177-4B | 8 | 46.4 | 20.9 | 22.5 | 55.6 | red |
| D100751-177-5A | 6 | 45.1 | 18.2 | 20.6 | 52.8 | red- |
| | | | | | | brown |
| D100751-177-5B | 6 | 44.8 | 20.1 | 22.2 | 53.9 | red- |
| | | | | | | brown |
| D100751-177-6A | 4 | 43.5 | 16.0 | 18.7 | 49.9 | brown |
| D100751-177-6B | 4 | 43.7 | 15.2 | 18.3 | 49.7 | brown |
| D100751-177-7A | 2 | 42.6 | 11.6 | 16.3 | 47.0 | brown |
| D100751-177-7B | 2 | 40.4 | 11.9 | 16.0 | 45.0 | brown |
| D100751-177 | 2 | 49.0 | 10.5 | 16.2 | 27.2 | brown |
| long tress | | | | | | |

### Example 7

### Post-Treatment of Colored Hair with Polymer Solutions to Enhance Retention of Hair Color Upon Washing

Peptides HC1048 (SEQ ID NO: 195), HC1049 (SEQ ID NO: 196), and HC1050 (SEQ ID NO: 197) were variations of peptide HC910 (SEQ ID NO: 179). Each has an N-terminal HP2 sequence (SEQ ID NO: 149) and a C-terminal MEA4 (SEQ ID NO: 165) sequence as hair-binding domains (sequences listed in Table 1), and a rationally-designed pigment-binding domain in between containing 24 positively-charged lysine residues. However, HC1048 (SEQ ID NO: 195), HC1049 (SEQ ID NO: 196), and HC1050 (SEQ ID NO: 197) differ slightly from each other in their pigment-binding domains (Table 10). These peptides were used to form adducts with silica-coated red iron oxide pigment using the same procedure outlined in Example 2.

After formation, adducts were applied to hair for hair coloring using the procedure described in Example 3. Each adduct was used to color ten hair tresses. After the hair tresses were rinsed with tap water and blotted with paper towels, they were placed into fresh tubes for different post-treatments as indicated in Table 11: polyallylamine hydrochloride (PAH) was from Sigma-Aldrich (catalog #283223), polyethyleneimine (PEI) was from J. T. Baker (catalog #U230-08), poly(diallyldimethylammonium chloride) (PDAC) was from Sigma-Aldrich (catalog #409014), chitosan was from Sigma-Aldrich (catalog #44887-7). These polymers were dissolved in 10 mM MES buffer pH 5 supplemented with 150 mM NaCl. The polymer concentrations used are listed in Table 11. Two colored hair tresses of each type were subjected to each kind of polymeric post-treatment as follows: to each tress, 0.8 mL of post-treatment solution was added, then the tube containing the tress and polymer solution was rotated end-over-end for 15 minutes. Afterwards, the tresses were removed and rinsed with tap water for 15 seconds per side with very mild gloved-finger embrocation. The tresses were blotted with paper towels, and then air-dried. L*, a*, b* color measurements were taken for each dried hair tress to quantify initial color uptake. L*, a*, b* reference values for untreated natural white hair were also measured for use in ΔE color difference calculations, ΔE=((L*−L*_{ref})²+(a*−a*_{ref})²+(b*−b*_{ref})²)^{0.5}. After initial color uptake readings, all hair tresses were subjected to 5 bead embrocation cycles in 2% SLES using the same procedure described in Example 3.

This example demonstrates that the use of polycation solutions as post-treatments for the adduct-colored tresses dramatically enhanced color (ΔE) retention after 5 wash cycles across the board, from ˜35% retention without post-treatment to 50-75% retention with polycation post-treatment after 5 cycles of bead embrocation. PAH effected the greatest improvement in color retention among the tested polymers.

TABLE 10
Sequence details for peptides HC1048, HC1049,
and HC1050.

| SEQ | | |
|---|---|---|
| ID | Peptide | |
| NO: | Name | Sequence Description |
| 195 | HC1048 | PS-HP2-(PK)₆FYF(PK)₆FYF(PK)₆FYF(PK)₆- |
| | | MEA4 |
| 196 | HC1049 | PS-HP2-(PK)₁₂VVI(PK)₆VVI(PK)₆-MEA4 |
| 197 | HC1050 | PS-HP2-(GK)₁₂VWL(PK)₆VWL(PK)₆-MEA4 |

TABLE 11
Color uptake and color retention for adduct-treated hair with and
without polymeric post-treatment.

| | | | | | % |
|---|---|---|---|---|---|
| | | | | | retention |
| Peptide | | | | | of |
| Name | | Mean ΔE- | | | deposition |
| (SEQ ID | | color | Mean ΔE- | Mean ΔE- | ΔE after |
| NO.) | Post-Treatment | deposition | ec1 | ec5 | ec5 |
| HC1048 | none | 36.6 | 23.9 | 12.1 | 33.0 |
| (Seq ID. | 0.5% PAH | 32.5 | 30.0 | 24.4 | 75.1 |
| No. 195) | 0.5% PEI | 33.5 | 30.7 | 22.1 | 65.9 |
| | 0.5% PDAC | 32.1 | 28.5 | 19.7 | 61.4 |
| | 0.1% chitosan | 34.9 | 29.7 | 21.2 | 60.9 |
| HC1049 | none | 37.1 | 25.6 | 12.7 | 34.2 |
| (Seq ID. | 0.5% PAH | 35.0 | 32.5 | 24.5 | 70.1 |
| No. 196) | 0.5% PEI | 34.5 | 30.6 | 21.5 | 62.4 |
| | 0.5% PDAC | 32.7 | 28.7 | 17.4 | 53.2 |
| | 0.1% chitosan | 34.6 | 29.6 | 21.0 | 60.7 |
| HC1050 | none | 39.3 | 27.0 | 13.9 | 35.3 |
| (Seq ID. | 0.5% PAH | 34.1 | 30.9 | 21.6 | 63.4 |
| No. 197) | 0.5% PEI | 33.4 | 30.7 | 22.1 | 66.1 |
| | 0.5% PDAC | 33.0 | 29.2 | 17.4 | 52.7 |
| | 0.1% chitosan | 34.1 | 30.1 | 21.4 | 63.0 |

What is claimed is:

1. A peptide-particulate benefit agent adduct comprising:
a) a particulate benefit agent; and
b) a peptide of having the general structure of
A1-(S1)ₚ-(X1-Y)ₙ—(X2)ₘ-(S2)_{q}-A2 or
A1-(S1)ₚ-(X1)ₘ-(Y—X2)ₙ-(S2)_{q}-A2
wherein,
A1 and A2 are binding domains having affinity to a body surface; wherein both A1 and A2 independently consist of 1 to 3 body surface-binding peptides (BSBP); each BSBP independently ranging from 7 to 60 amino acids in length and have affinity for the same body surface;
S1 and S2 are optional peptide spacers comprising 1 to 30 amino acids in length wherein the spacers contain less than 30 mol % charged amino acids
X1 and X2 are charged amino acid blocks; wherein X1 and X2 do not consist of net opposite charges; wherein X1 and X2 are independently 6 to 36 amino acids in length having 3 to 18 charged amino acids;
Y is a hydrophobic amino acid block comprising 3 to 10 contiguous hydrophobic amino acids;
m is an integer ranging from 0 to 10;
p and q are integers independently ranging from 0 to 3; and
n is an integer ranging from 1 to 50; and
wherein average particle size of the peptide-particulate benefit agent adduct is between 0.010 μm and 75 μm

2. The peptide-particulate benefit agent adduct of claim 1 where the charge amino acids in X1 and X2 are positively charged amino acids selected from the group consisting of arginine, lysine, and histidine.

3. The peptide-particulate benefit agent adduct of claim 2 wherein in the sum of positively charged amino acids in X1 and X2 is at least 12.

4. The peptide-particulate benefit agent adduct of claim 2 or claim 3 wherein the positively charged amino acids within X1 and X2 are separated by a non-charged amino acid.

5. The peptide-particulate benefit agent adduct of claim 4 wherein the non-charged amino acid separating the positively charged amino acids is glycine, proline, or a combination thereof.

6. The peptide-particulate benefit agent adduct of claim 1, wherein the benefit agent is a sunscreen agent, conditioning agent, encapsulated fragrance, antimicrobial, antidandruff, antifungal, odor control agent, encapsulated bioactive agent, hair removal agent, anti-acne agent, or coloring agent.

7. The peptide-particulate benefit agent adduct of claim 6, wherein the coloring agent is a pigment, colored particle, or a combination thereof.

8. The peptide-particulate benefit agent adduct of claim 1, wherein the body surface is hair, skin, nail, teeth, or an oral cavity tissue.

9. A stable dispersion comprising a stably-dispersed peptide-particulate benefit agent adduct of claim 1.

10. The stable dispersion of claim 9 where the stable dispersion is charge stabilized.

11. The stable dispersion of claim 10 wherein the peptide-particulate benefit agent adduct has a zeta potential absolute value of at least 20 mV.

12. The stable dispersion of claim 9 wherein the stable dispersion is sterically stabilized.

13. The stable dispersion of claim 9 wherein the stable dispersion further comprises a dispersant.

14. The stable dispersion of claim 13 wherein the dispersant is an ionic dispersant.

15. A method of forming a charge stabilized peptide-particulate benefit agent adduct comprising,
a) providing
1) a particulate benefit agent having average particle size between 0.010 μm and 75 μm;
2) the peptide of claim 1;
b) contacting the particulate benefit agent and the peptide in an aqueous medium under conditions suitable for forming a peptide-particulate benefit agent adduct; and
c) altering the pH of the aqueous medium until the absolute value of the zeta potential of the peptide-particulate benefit agent adduct is at least 20 mV.

16. A method of applying a benefit agent to a body surface, comprising,
a) contacting a body surface with a composition comprising a population of the peptide-particulate benefit agent adduct of claim 1 under conditions whereby a portion of the population of the peptide-particulate benefit agent adduct durably binds non-covalently to the body surface;
b) optionally, washing the body surface to remove non-durably bound peptide-particulate benefit agent adduct from the body surface;
c) optionally repeating steps (a) and (b).

17. The method of claim 16 wherein the particulate benefit agent comprises a pigment, a colored particle or a mixture thereof.

18. The method of claim 16 or claim 17, further comprising contacting the body surface with a cationic polymer after contacting the body surface with the peptide-particulate benefit agent adduct.

19. The method according to claim 17 wherein the composition comprising the population of the peptide-particulate benefit agent adduct is a mixture of adducts comprising 2 or more different pigments, colored particles, or combinations thereof.


SEQUENCE LISTING (199 sequences)

SEQ ID NO: 1 - protein, 8 aa, Artificial Sequence
  synthetic construct
LESTPKMK

SEQ ID NO: 2 - protein, 7 aa, Artificial Sequence
  synthetic construct
FTQSLPR

SEQ ID NO: 3 - protein, 12 aa, Artificial Sequence
  synthetic construct
SVSVGMKPSPRP

SEQ ID NO: 4 - protein, 12 aa, Artificial Sequence
  synthetic construct
LDVESYKGTSMP

SEQ ID NO: 5 - protein, 12 aa, Artificial Sequence
  synthetic construct
RVPNKTVTVDGA

SEQ ID NO: 6 - protein, 12 aa, Artificial Sequence
  synthetic construct
DRHKSKYSSTKS

SEQ ID NO: 7 - protein, 12 aa, Artificial Sequence
  synthetic construct
KNFPQQKEFPLS

SEQ ID NO: 8 - protein, 12 aa, Artificial Sequence
  synthetic construct
QRNSPPAMSRRD

SEQ ID NO: 9 - protein, 12 aa, Artificial Sequence
  synthetic construct
TRKPNMPHGQYL

SEQ ID NO: 10 - protein, 12 aa, Artificial Sequence
  synthetic construct
KPPHLAKLPFTT

SEQ ID NO: 11 - protein, 12 aa, Artificial Sequence
  synthetic construct
NKRPPTSHRIHA

SEQ ID NO: 12 - protein, 12 aa, Artificial Sequence
  synthetic construct
NLPRYQPPCKPL

SEQ ID NO: 13 - protein, 12 aa, Artificial Sequence
  synthetic construct
RPPWKKPIPPSE

SEQ ID NO: 14 - protein, 12 aa, Artificial Sequence
  synthetic construct
RQRPKDHFFSRP

SEQ ID NO: 15 - protein, 12 aa, Artificial Sequence
  synthetic construct
SVPNKXVTVDGX

SEQ ID NO: 16 - protein, 12 aa, Artificial Sequence
  synthetic construct
TTKWRHRAPVSP

SEQ ID NO: 17 - protein, 12 aa, Artificial Sequence
  synthetic construct
WLGKNRIKPRAS

SEQ ID NO: 18 - protein, 12 aa, Artificial Sequence
  synthetic construct
SNFKTPLPLTQS

SEQ ID NO: 19 - protein, 12 aa, Artificial Sequence
  synthetic construct
KELQTRNVVQRE

SEQ ID NO: 20 - protein, 12 aa, Artificial Sequence
  synthetic construct
TPTANQFTQSVP

SEQ ID NO: 21 - protein, 12 aa, Artificial Sequence
  synthetic construct
AAGLSQKHERNR

SEQ ID NO: 22 - protein, 12 aa, Artificial Sequence
  synthetic construct
ETVHQTPLSDRP

SEQ ID NO: 23 - protein, 12 aa, Artificial Sequence
  synthetic construct
LPALHIQRHPRM

SEQ ID NO: 24 - protein, 12 aa, Artificial Sequence
  synthetic construct
QPSHSQSHNLRS

SEQ ID NO: 25 - protein, 12 aa, Artificial Sequence
  synthetic construct
RGSQKSKPPRPP

SEQ ID NO: 26 - protein, 12 aa, Artificial Sequence
  synthetic construct
THTQKTPLLYYH

SEQ ID NO: 27 - protein, 12 aa, Artificial Sequence
  synthetic construct
TKGSSQAILKST

SEQ ID NO: 28 - protein, 7 aa, Artificial Sequence
  synthetic construct
DLHTVYH

SEQ ID NO: 29 - protein, 7 aa, Artificial Sequence
  synthetic construct
HIKPPTR

SEQ ID NO: 30 - protein, 7 aa, Artificial Sequence
  synthetic construct
HPVWPAI

SEQ ID NO: 31 - protein, 7 aa, Artificial Sequence
  synthetic construct
MPLYYLQ

SEQ ID NO: 32 - protein, 26 aa, Artificial Sequence
  synthetic construct
HLTVPWRGGGSAVPFYSHSQITLPNH

SEQ ID NO: 33 - protein, 41 aa, Artificial Sequence
  synthetic construct
GPHDTSSGGVRPNLHHTSKKEKRENRKVPFYSHSVTSRGNV

SEQ ID NO: 34 - protein, 7 aa, Artificial Sequence
  synthetic construct
KHPTYRQ

SEQ ID NO: 35 - protein, 7 aa, Artificial Sequence
  synthetic construct
HPMSAPR

SEQ ID NO: 36 - protein, 7 aa, Artificial Sequence
  synthetic construct
MPKYYLQ

SEQ ID NO: 37 - protein, 7 aa, Artificial Sequence
  synthetic construct
MHAHSIA

SEQ ID NO: 38 - protein, 7 aa, Artificial Sequence
  synthetic construct
TAATTSP

SEQ ID NO: 39 - protein, 7 aa, Artificial Sequence
  synthetic construct
LGIPQNL

SEQ ID NO: 40 - protein, 12 aa, Artificial Sequence
  synthetic construct
AKPISQHLQRGS

SEQ ID NO: 41 - protein, 12 aa, Artificial Sequence
  synthetic construct
APPTPAAASATT

SEQ ID NO: 42 - protein, 12 aa, Artificial Sequence
  synthetic construct
DPTEGARRTIMT

SEQ ID NO: 43 - protein, 12 aa, Artificial Sequence
  synthetic construct
EQISGSLVAAPW

SEQ ID NO: 44 - protein, 12 aa, Artificial Sequence
  synthetic construct
LDTSFPPVPFHA

SEQ ID NO: 45 - protein, 11 aa, Artificial Sequence
  synthetic construct
LPRIANTWSPS

SEQ ID NO: 46 - protein, 12 aa, Artificial Sequence
  synthetic construct
RTNAADHPAAVT

SEQ ID NO: 47 - protein, 12 aa, Artificial Sequence
  synthetic construct
SLNWVTIPGPKI

SEQ ID NO: 48 - protein, 12 aa, Artificial Sequence
  synthetic construct
TDMQAPTKSYSN

SEQ ID NO: 49 - protein, 12 aa, Artificial Sequence
  synthetic construct
TIMTKSPSLSCG

SEQ ID NO: 50 - protein, 12 aa, Artificial Sequence
  synthetic construct
TPALDGLRQPLR

SEQ ID NO: 51 - protein, 12 aa, Artificial Sequence
  synthetic construct
TYPASRLPLLAP

SEQ ID NO: 52 - protein, 12 aa, Artificial Sequence
  synthetic construct
AKTHKHPAPSYS

SEQ ID NO: 53 - protein, 12 aa, Artificial Sequence
  synthetic construct
YPSFSPTYRPAF

SEQ ID NO: 54 - protein, 12 aa, Artificial Sequence
  synthetic construct
TDPTPFSISPER

SEQ ID NO: 55 - protein, 20 aa, Artificial Sequence
  synthetic construct
CAAGCCTCAGCGACCGAATA

SEQ ID NO: 56 - protein, 12 aa, Artificial Sequence
  synthetic construct
WHDKPQNSSKST

SEQ ID NO: 57 - protein, 12 aa, Artificial Sequence
  synthetic construct
NEVPARNAPWLV

SEQ ID NO: 58 - protein, 13 aa, Artificial Sequence
  synthetic construct
NSPGYQADSVAIG

SEQ ID NO: 59 - protein, 12 aa, Artificial Sequence
  synthetic construct
TQDSAQKSPSPL

SEQ ID NO: 60 - protein, 12 aa, Artificial Sequence
  synthetic construct
ALPRIANTWSPS

SEQ ID NO: 61 - protein, 12 aa, Artificial Sequence
  synthetic construct
TPFHSPENAPGS

SEQ ID NO: 62 - DNA, 20 bp, Artificial Sequence
  Primer
ccctcatagttagcgtaacg

SEQ ID NO: 63 - protein, 16 aa, Artificial Sequence
  synthetic construct
RTNAADHPAAVTGGGC

SEQ ID NO: 64 - protein, 8 aa, Artificial Sequence
  Caspase 3 cleavage site
LESGDEVD

SEQ ID NO: 65 - protein, 12 aa, Artificial Sequence
  synthetic construct
TPPELLHGDPRS

SEQ ID NO: 66 - protein, 12 aa, Artificial Sequence
  synthetic construct
TPPTNVLMLATK

SEQ ID NO: 67 - protein, 7 aa, Artificial Sequence
  synthetic construct
NTSQLST

SEQ ID NO: 68 - protein, 14 aa, Artificial Sequence
  synthetic construct
RTNAADHPAAVTKC

SEQ ID NO: 69 - protein, 14 aa, Artificial Sequence
  synthetic construct
ALPRIANTWSPSKC

SEQ ID NO: 70 - protein, 14 aa, Artificial Sequence
  synthetic construct
TPPELLHGDPRSKC

SEQ ID NO: 71 - protein, 14 aa, Artificial Sequence
  synthetic construct
TPFHSPENAPGSKC

SEQ ID NO: 72 - protein, 15 aa, Artificial Sequence
  synthetic construct
STLHKYKSQDPTPHH

SEQ ID NO: 73 - protein, 7 aa, Artificial Sequence
  synthetic construct
NTPKENW

SEQ ID NO: 74 - protein, 7 aa, Artificial Sequence
  synthetic construct
NTPASNR

SEQ ID NO: 75 - protein, 7 aa, Artificial Sequence
  synthetic construct
PRGMLST

SEQ ID NO: 76 - protein, 7 aa, Artificial Sequence
  synthetic construct
PPTYLST

SEQ ID NO: 77 - protein, 12 aa, Artificial Sequence
  synthetic construct
TIPTHRQHDYRS

SEQ ID NO: 78 - protein, 7 aa, Artificial Sequence
  synthetic construct
TPPTHRL

SEQ ID NO: 79 - protein, 7 aa, Artificial Sequence
  synthetic construct
LPTMSTP

SEQ ID NO: 80 - protein, 7 aa, Artificial Sequence
  synthetic construct
LGTNSTP

SEQ ID NO: 81 - protein, 12 aa, Artificial Sequence
  synthetic construct
TPLTGSTNLLSS

SEQ ID NO: 82 - protein, 7 aa, Artificial Sequence
  synthetic construct
TPLTKET

SEQ ID NO: 83 - protein, 7 aa, Artificial Sequence
  synthetic construct
QQSHNPP

SEQ ID NO: 84 - protein, 7 aa, Artificial Sequence
  synthetic construct
TQPHNPP

SEQ ID NO: 85 - protein, 12 aa, Artificial Sequence
  synthetic construct
STNLLRTSTVHP

SEQ ID NO: 86 - protein, 12 aa, Artificial Sequence
  synthetic construct
HTQPSYSSTNLF

SEQ ID NO: 87 - protein, 7 aa, Artificial Sequence
  synthetic construct
SLLSSHA

SEQ ID NO: 88 - protein, 12 aa, Artificial Sequence
  synthetic construct
QQSSISLSSHAV

SEQ ID NO: 89 - protein, 7 aa, Artificial Sequence
  synthetic construct
NASPSSL

SEQ ID NO: 90 - protein, 7 aa, Artificial Sequence
  synthetic construct
HSPSSLR

SEQ ID NO: 91 - protein, 7 aa, Artificial Sequence
  synthetic construct
KXSHHTH

SEQ ID NO: 92 - protein, 7 aa, Artificial Sequence
  synthetic construct
EXSHHTH

SEQ ID NO: 93 - protein, 7 aa, Artificial Sequence
  synthetic construct
LESTSLL

SEQ ID NO: 94 - protein, 7 aa, Artificial Sequence
  synthetic construct
TPLTKET

SEQ ID NO: 95 - protein, 7 aa, Artificial Sequence
  synthetic construct
KQSHNPP

SEQ ID NO: 96 - protein, 12 aa, Artificial Sequence
  synthetic construct
KQATFPPNPTAY

SEQ ID NO: 97 - protein, 12 aa, Artificial Sequence
  synthetic construct
HGHMVSTSQLSI

SEQ ID NO: 98 - protein, 7 aa, Artificial Sequence
  synthetic construct
LSPSRMK

SEQ ID NO: 99 - protein, 7 aa, Artificial Sequence
  synthetic construct
LPIPRMK

SEQ ID NO: 100 - protein, 7 aa, Artificial Sequence
  synthetic construct
HQRPYLT

SEQ ID NO: 101 - protein, 7 aa, Artificial Sequence
  synthetic construct
FPPLLRL

SEQ ID NO: 102 - protein, 12 aa, Artificial Sequence
  synthetic construct
KRGRHKRPKRHK

SEQ ID NO: 103 - protein, 7 aa, Artificial Sequence
  synthetic construct
RLLRLLR

SEQ ID NO: 104 - protein, 12 aa, Artificial Sequence
  synthetic construct
HKPRGGRKKALH

SEQ ID NO: 105 - protein, 18 aa, Artificial Sequence
  synthetic construct
KPRPPHGKKHRPKHRPKK

SEQ ID NO: 106 - protein, 18 aa, Artificial Sequence
  synthetic construct
RGRPKKGHGKRPGHRARK

SEQ ID NO: 107 - protein, 7 aa, Artificial Sequence
  synthetic construct
MPPPLMQ

SEQ ID NO: 108 - protein, 7 aa, Artificial Sequence
  synthetic construct
FHENWPS

SEQ ID NO: 109 - protein, 12 aa, Artificial Sequence
  synthetic construct
RTAPTTPLLLSL

SEQ ID NO: 110 - protein, 12 aa, Artificial Sequence
  synthetic construct
WHLSWSPVPLPT

SEQ ID NO: 111 - protein, 7 aa, Artificial Sequence
  synthetic construct
PHARLVG

SEQ ID NO: 112 - protein, 14 aa, Artificial Sequence
  synthetic construct
NIPYHHPNIPYHHP

SEQ ID NO: 113 - protein, 7 aa, Artificial Sequence
  synthetic construct
TTMPAIP

SEQ ID NO: 114 - protein, 7 aa, Artificial Sequence
  synthetic construct
HNLPPRS

SEQ ID NO: 115 - protein, 12 aa, Artificial Sequence
  synthetic construct
AHKTQMGVRQPA

SEQ ID NO: 116 - protein, 12 aa, Artificial Sequence
  synthetic construct
ADNVQMGVSHTP

SEQ ID NO: 117 - protein, 12 aa, Artificial Sequence
  synthetic construct
AHNAQMGVSHPP

SEQ ID NO: 118 - protein, 12 aa, Artificial Sequence
  synthetic construct
ADYVGMGVSHRP

SEQ ID NO: 119 - protein, 12 aa, Artificial Sequence
  synthetic construct
SVSVGMKPSPRP

SEQ ID NO: 120 - protein, 7 aa, Artificial Sequence
  synthetic construct
YPNTALV

SEQ ID NO: 121 - protein, 7 aa, Artificial Sequence
  synthetic construct
VATRIVS

SEQ ID NO: 122 - protein, 12 aa, Artificial Sequence
  synthetic construct
HSLKNSMLTVMA

SEQ ID NO: 123 - protein, 7 aa, Artificial Sequence
  synthetic construct
NYPTQAP

SEQ ID NO: 124 - protein, 7 aa, Artificial Sequence
  synthetic construct
KCCYSVG

SEQ ID NO: 125 - protein, 12 aa, Artificial Sequence
  synthetic construct
RHDLNTWLPPVK

SEQ ID NO: 126 - protein, 12 aa, artificial sequence
  synthetic construct
EISLPAKLPSAS

SEQ ID NO: 127 - protein, 12 aa, Artificial Sequence
  synthetic construct
YVCEGIHPCPRP

SEQ ID NO: 128 - protein, 12 aa, Artificial Sequence
  synthetic construct
SDYVGMRPSPRH

SEQ ID NO: 129 - protein, 12 aa, Artificial Sequence
  synthetic construct
SDYVGMRLSPSQ

SEQ ID NO: 130 - protein, 12 aa, Artificial Sequence
  synthetic construct
SVSVGIQPSPRP

SEQ ID NO: 131 - protein, 12 aa, Artificial Sequence
  synthetic construct
YVSVGIKPSPRP

SEQ ID NO: 132 - protein, 37 aa, Artificial Sequence
  synthetic construct
TSTSKASTTTTSSKTTTTSSKTTTTTSKTSTTSSSST

SEQ ID NO: 133 - protein, 22 aa, Artificial Sequence
  synthetic construct
GQGGYGGLGSQGAGRGGLGGQG

SEQ ID NO: 134 - protein, 10 aa, Artificial Sequence
  synthetic construct
GPGGYGPGQQ

SEQ ID NO: 135 - protein, 12 aa, Artificial Sequence
  synthetic construct
GMPAMHWIHPFA

SEQ ID NO: 136 - protein, 15 aa, Artificial Sequence
  synthetic construct
HDHKNQKETHQRHAA

SEQ ID NO: 137 - protein, 20 aa, Artificial Sequence
  synthetic construct
HNHMQERYTDPQHSPSVNGL

SEQ ID NO: 138 - protein, 20 aa, Artificial Sequence
  synthetic construct
TAEIQSSKNPNPHPQRSWTN

SEQ ID NO: 139 - protein, 21 aa, Artificial sequence
  synthetic construct
HTNDNGQSTPRRDPPAFQRKK

SEQ ID NO: 140 - protein, 16 aa, artificial sequence
  synthetic construct
YKHERHYSQPLKVRHK

SEQ ID NO: 141 - protein, 12 aa, artificial sequence
  synthetic construct
HPPMNASHPHMH

SEQ ID NO: 142 - protein, 16 aa, artificial sequence
  synthetic construct
SDETGPQIPHRRATWK

SEQ ID NO: 143 - protein, 12 aa, artificial sequence
  synthetic construct
HKLPSASRHHFH

SEQ ID NO: 144 - protein, 13 aa, artificial sequence
  synthetic construct
IPWWNIRAPLNAK

SEQ ID NO: 145 - protein, 13 aa, artificial sequence
  synthetic construct
TPPELLHGDPRSK

SEQ ID NO: 146 - protein, 15 aa, artificial sequence
  synthetic construct
HNYHYPHTGHMAHSA

SEQ ID NO: 147 - protein, 15 aa, artificial sequence
  synthetic construct
HNNHYPHGGHMAHAA

SEQ ID NO: 148 - protein, 15 aa, artificial sequence
  synthetic construct
HQNHQNHQNHQNHQN

SEQ ID NO: 149 - protein, 20 aa, artificial sequence
  synthetic construct
AQSQLPDKHSGLHERAPQRY

SEQ ID NO: 150 - protein, 15 aa, artificial sequence
  synthetic construct
HQLHQLHQLHQLHQL

SEQ ID NO: 151 - protein, 19 aa, artificial sequence
  synthetic construct
HHDRAEPRGMAATLAQTIK

SEQ ID NO: 152 - protein, 12 aa, artificial sequence
  synthetic construct
HTKHSHTSPPPL

SEQ ID NO: 153 - protein, 21 aa, artificial sequence
  synthetic construct
LNSMSDKHHGHQNTATRNQHK

SEQ ID NO: 154 - protein, 15 aa, artificial sequence
  synthetic construct
FASAHHTHTHHGAGF

SEQ ID NO: 155 - protein, 15 aa, artificial sequence
  synthetic construct
HSLHSLHSLHSLHSL

SEQ ID NO: 156 - protein, 15 aa, artificial sequence
  synthetic construct
HGLHGLHGLHGLHGL

SEQ ID NO: 157 - protein, 12 aa, artificial sequence
  synthetic construct
HVSHFHASRHER

SEQ ID NO: 158 - protein, 12 aa, artificial sequence
  synthetic construct
HVSHHATGHTHT

SEQ ID NO: 159 - protein, 15 aa, artificial sequence
  synthetic construct
HGHGHGAGAAHGHGH

SEQ ID NO: 160 - protein, 29 aa, artificial sequence
  synthetic construct
GPEEAAKKEEAAKKEEAAKKEEAAKKPAK

SEQ ID NO: 161 - protein, 15 aa, artificial sequence
  synthetic construct
HQAHQAHQAHQAHQA

SEQ ID NO: 162 - protein, 15 aa, artificial sequence
  synthetic construct
HHGTHHNATKQKNHV

SEQ ID NO: 163 - protein, 21 aa, artificial sequence
  synthetic construct
DHNNRQHAVEVRENKTHTARK

SEQ ID NO: 164 - protein, 12 aa, artificial sequence
  synthetic construct
HGSKANHPHIRA

SEQ ID NO: 165 - protein, 20 aa, artificial sequence
  synthetic construct
HINKTNPHQGNHHSEKTQRQ

SEQ ID NO: 166 - protein, 15 aa, artificial sequence
  synthetic construct
AHHASTGGTSSAHHA

SEQ ID NO: 167 - protein, 39 aa, artificial sequence
  synthetic construct
HHGTHHNATKQKNHVGGSGPGSGGHHGTHHNATKQKNHV

SEQ ID NO: 168 - protein, 15 aa, artificial sequence
  synthetic construct
GKGKGKGKGKGKGKG

SEQ ID NO: 169 - protein, 15 aa, artificial sequence
  synthetic construct
HSGHSGHSGHSGHSG

SEQ ID NO: 170 - protein, 16 aa, artificial sequence
  synthetic construct
HDHKNQKETHQRHAAK

SEQ ID NO: 171 - protein, 15 aa, artificial sequence
  synthetic construct
AVAGKGKGKGKGAVA

SEQ ID NO: 172 - protein, 15 aa, artificial sequence
  synthetic construct
AKAKPAKAKPAKAKA

SEQ ID NO: 173 - protein, 28 aa, artificial sequence
  synthetic construct
PWRRRIVWRFMRNHALASMLWLSVSTVK

SEQ ID NO: 174 - protein, 12 aa, artificial sequence
  synthetic construct
RKKRKKFYFYFY

SEQ ID NO: 175 - protein, 38 aa, artificial sequence
  synthetic construct
GPEPEPEPEPIPEPPKEAPVVIEKPKPKPKPKPKPPAK

SEQ ID NO: 176 - protein, 43 aa, artificial sequence
  synthetic construct
GPEPEPEPEPIPEPPKEAPVVIEKPKPKPKPKPKPPAHHHHHH

SEQ ID NO: 177 - protein, 95 aa, artificial sequence
  synthetic construct
PSAQSQLPDKHSGLHERAPQRYPKPKPKPKPKPKAPVVIPKPKPKPKPKPKPKPKPKPKPKPKAPVVIPKPKPKPKPKPKHDHKNQKETHQRHAA

SEQ ID NO: 178 - protein, 113 aa, artificial sequence
  synthetic construct
PSAQSQLPDKHSGLHERAPQRYGSSGPGSGSPKPKPKPKPKPKAPVVIPKPKPKPKPKPKPKPKPKPKPKPKAPVVIPKPKPKPKPKPKGSSGPGSGSHDHKNQKETHQRHAA

SEQ ID NO: 179 - protein, 100 aa, artificial sequence
  synthetic construct
PSAQSQLPDKHSGLHERAPQRYPKPKPKPKPKPKAPVVIPKPKPKPKPKPKPKPKPKPKPKPKAPVVIPKPKPKPKPKPKHINKTNPHQGNHHSEKTQRQ

SEQ ID NO: 180 - protein, 136 aa, artificial sequence
  synthetic construct
PSAQSQLPDKHSGLHERAPQRYGSSGPGSGSSGPGSGSSGPKPKPKPKPKPKAPVVIPKPKPKPKPKPKPKPKPKPKPKPKAPVVIPKPKPKPKPKPKGSSGPGSGSSGPGSGSSGHINKTNPHQGNHHSEKTQRQ

SEQ ID NO: 181 - protein, 95 aa, artificial sequence
  synthetic construct
PSHDHKNQKETHQRHAAGKGKGKGKGKGKAPVVIGKGKGKGKGKGKGKGKGKGKGKGKAPVVIGKGKGKGKGKGKHINKTNPHQGNHHSEKTQRQ

SEQ ID NO: 182 - protein, 150 aa, artificial sequence
  synthetic construct
PSHDHKNQKETHQRHAAGSSGPGSGSSGPGSGSSGPGSGSSGGKGKGKGKGKGKAPVVIGKGKGKGKGKGKGKGKGKGKGKGKAPVVIGKGKGKGKGKGKGSSGPGSGSSGPGSGSSGPGSGSSGPGSSGHINKTNPHQGNHHSEKTQRQ

SEQ ID NO: 183 - protein, 77 aa, artificial sequence
  synthetic construct
PSAQSQLPDKHSGLHERAPQRYGPGAGGAGGSGGSGAGGAGGSGGSPAHDHKNQKETHQRHAAGSGGGGSPHHHHHH

SEQ ID NO: 184 - protein, 88 aa, artificial sequence
  synthetic construct
PSAQSQLPDKHSGLHERAPQRYGPEPEPEPEPIPEPPKEAPVVIEKPKPKPKPKPKPPAHDHKNQKETHQRHAAGSGGGGSPHHHHHH

SEQ ID NO: 185 - protein, 129 aa, artificial sequence
  synthetic construct
PSAQSQLPDKHSGLHERAPQRYGPEPEPEPEPIPEPPKEAPVVIEKPKPKPKPKPKPPAHDHKNQKETHQRHAAGSGGGGSPWAPEKDHMQLMKGKGKGKGKGKGKGKGKGKGKGWAPEKDHMQLMKGK

SEQ ID NO: 186 - protein, 93 aa, artificial sequence
  synthetic construct
PRAQSQLPDKHSGLHERAPQRYGPEPEPEPEPIPEPPKEAPVVIEKPKPKPKPKPKPPAHINKTNPHQGNHHSEKTQRQGSGGGGSPHHHHHH

SEQ ID NO: 187 - protein, 54 aa, artificial sequence
  synthetic construct
PSAQSQLPDKHSGLHERAPQRYPKPKPKPKPKPKHINKTNPHQGNHHSEKTQRQ

SEQ ID NO: 188 - protein, 66 aa, artificial sequence
  synthetic construct
PSAQSQLPDKHSGLHERAPQRYPKPKPKPKPKPKPKPKPKPKPKPKHINKTNPHQGNHHSEKTQRQ

SEQ ID NO: 189 - protein, 78 aa, artificial peptide
  synthetic construct
PSAQSQLPDKHSGLHERAPQRYPKPKPKPKPKPKPKPKPKPKPKPKPKPKPKPKPKPKHINKTNPHQGNHHSEKTQRQ

SEQ ID NO: 190 - protein, 80 aa, artificial sequence
  synthetic construct
PSAQSQLPDKHSGLHERAPQRYPKPKPKPKPKPKWPKPKPKPKPKPKVPKPKPKPKPKPKHINKTNPHQGNHHSEKTQRQ

SEQ ID NO: 191 - protein, 92 aa, artificial sequence
  synthetic construct
PSAQSQLPDKHSGLHERAPQRYPKPKPKPKPKPKWPKPKPKPKPKPKPKPKPKPKPKPKVPKPKPKPKPKPKHINKTNPHQGNHHSEKTQRQ

SEQ ID NO: 192 - protein, 82 aa, artificial sequence
  synthetic construct
PSAQSQLPDKHSGLHERAPQRYPKPKPKPKPKPKWWPKPKPKPKPKPKVWPKPKPKPKPKPKHINKTNPHQGNHHSEKTQRQ

SEQ ID NO: 193 - protein, 99 aa, artificial sequence
  synthetic construct
PSAQSQLPDKHSGLHERAPQRYPKPKPKPKPKPKVVIPKPKPKPKPKPKVVIPKPKPKPKPKPKVVIPKPKPKPKPKPKHINKTNPHQGNHHSEKTQRQ

SEQ ID NO: 194 - protein, 132 aa, artificial sequence
  synthetic construct
PSAQSQLPDKHSGLHERAPQRYGPTTTTSSKTTTTSSKPAPKPKPKPKPKPKPKPKPKPKPKPKVVIPKPKPKPKPKPKVVIPKPKPKPKPKPKGPTTTTSSKTTTTSSKPAHINKTNPHQGNHHSEKTQRQ

SEQ ID NO: 195 - protein, 99 aa, artificial sequence
  synthetic construct
PSAQSQLPDKHSGLHERAPQRYPKPKPKPKPKPKFYFPKPKPKPKPKPKFYFPKPKPKPKPKPKFYFPKPKPKPKPKPKHINKTNPHQGNHHSEKTQRQ

SEQ ID NO: 196 - protein, 96 aa, artificial sequence
  synthetic construct
PSAQSQLPDKHSGLHERAPQRYPKPKPKPKPKPKPKPKPKPKPKPKVVIPKPKPKPKPKPKVVIPKPKPKPKPKPKHINKTNPHQGNHHSEKTQRQ

SEQ ID NO: 197 - protein, 96 aa, artificial sequence
  synthetic construct
PSAQSQLPDKHSGLHERAPQRYGKGKGKGKGKGKGKGKGKGKGKGKVWLPKPKPKPKPKPKVWLPKPKPKPKPKPKHINKTNPHQGNHHSEKTQRQ

SEQ ID NO: 198 - protein, 58 aa, artificial sequence
  synthetic construct
PKPKPKPKPKPKAPVVIPKPKPKPKPKPKPKPKPKPKPKPKAPVVIPKPKPKPKPKPK

SEQ ID NO: 199 - protein, 58 aa, artificial sequence
  synthetic construct
GKGKGKGKGKGKAPVVIGKGKGKGKGKGKGKGKGKGKGKGKAPVVIGKGKGKGKGKGK